(19) United States
(12) Patent Application Publication
(10) Pub. No.: US 2007/0172831 A1
(43) Pub. Date: Jul. 26, 2007

(54) Schizophrenia gene signatures and methods of using the same

(76) Inventors: C. Anthony Altar, Garrett Park, MD (US); Jeffrey A. Brockman, Frederick, MD (US); Vinod D. Charles, Silver Spring, MD (US); Linda Jurata, Poolesville, MD (US); Yury Bukhman, Mississauga (CA)

Correspondence Address: DARBY & DARBY P.C., P.O. BOX 770, Church Street Station, New York, NY, 10008-0770, US

(21) Appl. No.: 10/873,426

(22) Filed: Jun. 21, 2004

Related U.S. Application Data
(60) Provisional application No. 60/480,100, filed on Jun. 19, 2003.

(51) Int. Cl. C12Q 1/68

(52) U.S. Cl. 435/6

(57) ABSTRACT

Compositions and methods that are useful for the diagnosis and treatment of schizophrenia are provided. More specifically, “gene signatures” are described that are characteristic of schizophrenia in an individual. The specific classes of genes that can be identified from these signatures are useful in that they provide the basis for identification of novel therapeutic protein targets for the treatment of schizophrenia, and provide potential diagnostic markers for schizophrenia and markers for evaluating the therapeutic response to antipsychotic agents.

### CROSS-REFERENCE TO RELATED APPLICATIONS

This application claims the benefit of priority under 35 U.S.C. §119(e) of U.S. Provisional Patent Application Ser. No. 60/480,100, filed on Jun. 19, 2003. The contents of the priority application are hereby incorporated into the present disclosure by reference in their entirety.

### STATEMENT UNDER 37 C.F.R. §1.77(b)(4)

This application refers to a “Sequence Listing” listed below, which is provided as an electronic document on two identical compact discs, labeled “Copy 1” and “Copy 2.” These compact discs each contain the file named “100M970.ST25.pdf” (670,208 bytes, created on Jun. 21, 2004). Pursuant to 37 C.F.R. § 1.77(b)(4), the sequence listing on these compact disc is hereby incorporated by reference into the subject application.

### FIELD OF THE INVENTION

The present invention relates to compositions and methods that are useful for the diagnosis and treatment of schizophrenia. More specifically, the invention comprises sets of genes referrred to as “gene signatures” that are characteristic of schizophrenia in an individual. The set of genes marked by the signatures provide the basis for the identification of novel therapeutic protein targets for schizophrenia, as well as potential diagnostic markers for schizophrenia and markers for evaluating the therapeutic response to antipsychotic agents.

### BACKGROUND OF THE INVENTION

In order to facilitate reference to various journal articles, a listing of the articles is provided at the end of this specification. However, the listing or citation of these or other references does not constitute an admission that the reference(s) is(are) “prior art” to the present invention.

Schizophrenia is estimated to be prevalent in up to 1% of the population. While small molecule drugs are used to treat the disease, these drugs all exhibit side effects. In addition, many patients are or become resistant to these treatments. The mode of action for these drugs is thought to be through antagonist/agonist action of G protein coupled receptors that mediate neurotransmission. These small molecule-receptor interactions may also be responsible for the negative or side effects of these drugs as well. The major challenge in developing superior drugs that treat the root causes or impairments in schizophrenia is the lack of identified biochemical process targets that are aberrant in the disease.

Biochemical studies on post-mortem schizophrenic tissue have to date not provided a comprehensive set of such biochemical targets that are amenable to drug discovery. Several brain regions have been implicated in the pathophysiology of schizophrenia, particularly the hippocampus, frontal cortex, and temporal lobe (Tamminga et al., 1992; Benes, et al., 2002). Biochemical changes within these regions include decreases in neuronal size, increased cellular packing densities, distortions in neuronal orientation (Arnold & Trojanowski, 1996; Byne et al., 2002; Harrison, 1999), alterations in various neurotransmitter pathways and presynaptic components (Beasley et al., 2002; Benes, 2000). Changes include findings from positron emission tomography imaging studies, which have revealed abnormalities of regional cerebral blood flow (CBF) and glucose metabolism in the hippocampus and prefrontal cortex of schizophrenic patients (Tamminga et al., 1992; Dickey et al., 2002; McCarley et al., 1999; Kishimoto et al., 1998). At a cellular level, cortical interneurons, hippocampal dentate granule neurons, and CA3 pyramidal cells have been most strongly implicated as being different in schizophrenia or bipolar disease. Unfortunately, these morphological studies provide little information about potential functional impairments or routes for therapeutic intervention using drug discovery methods.

An alternative strategy is the comparison of gene expression profiles within defined neuron populations from the brains of normal and diseased patients. A single study has combined laser capture microdissection (LCM) with T7-based RNA amplification to obtain genomic expression profiles from a neuronal population, the rat dorsal root ganglion (Luo et al., 1999; Van Gelder et al., 1990; Eberwine et al., 1990). The only similar study in on brain tissues identified gene expression in single entorhinal cortical neurons in schizophrenic and normal cases (Hemby et al., 2002). A down-regulation of various G-protein-coupled receptor-signaling transcripts, glutamate receptor subunits, and synaptic proteins was seen in the schizophrenia cases.

The advent of microarray-based gene expression profiling has allowed several groups to identify CNS gene expression changes in schizophrenics. These studies have uniformly used frozen blocks of frontal cortex, and revealed alterations in genes that encode for proteins involved in synaptic signaling (Hemby et al., 2002; Mirnics et al., 2000), neurotransmitters (Vawter et al; Bahn et al. 2001), myelination (Hakak et al., 2001; Davis et al., 2003) and energy metabolism (Middleton et al., 2002). However, the presence of multiple cell types within the tissue blocks used in these studies may dilute and mask gene expression changes otherwise seen in specific cell populations. The impact of schizophrenia or any psychiatric disease on gene expression within hippocampal neurons remains unknown.

### SUMMARY OF THE INVENTION

The present invention provides novel “gene signatures” that are indicative of schizophrenia. Another embodiment of the invention comprises a method for diagnosing whether a patient has schizophrenia. In yet another embodiment, the invention comprises a method for monitoring a therapeutic response in an individual undergoing treatment for schizophrenia. In an alternative embodiment, the present invention provides kits for diagnosing schizophrenia in an individual. In another embodiment, the present invention describes measurement of gene expression profiles of neurons extracted from the hippocampal dentate gyrus or CA3 region of schizophrenic, bipolar, major depression patients and controls. Amplified antisense RNA (aRNA) prepared from these samples is analyzed, e.g., by cDNA microarrays to identify disease-specific changes in gene expression. The dentate granule cells and CA3 neurons reveal robust changes in gene expression in schizophrenia relative to controls. Most pronounced are decreases in macromolecular complexes involved in mitochondrial function and energy metabolism (NADH dehyrdogenase, malate dehyrdogenase, ubininol:cytochrome c reductase, succinate dehydrogenase, cytochrome c oxidase and ATP synthase) and proteasome function (proteasome subunits, ubiquitin, and proteasome-specific ATP synthase). Genes involved in synaptic transmission (syntaxin 8, syntenin, SNAP 25 and drebrin), neurite outgrowth, and cytoskeletal proteins (GAP-43, cadherin-like 22 and contactin and RAB 33-A) are also consistently decreased. These macromolecular-specific changes in gene expression in schizophrenia demonstrate highly statistically significant decreases in expression level between the normal and schizophrenic data sets.

A second example describes experiments in which gene expression profiles of neurons extracted from the hippocampal dentate gyrus of schizophrenic, bipolar, major depression patients and controls were measured. Amplified antisense RNA (aRNA) prepared from these samples is analyzed, e.g., by cDNA microarrays to identify disease-specific changes in gene expression. Again, the dentate granule cells reveal robust changes in gene expression in schizophrenia relative to controls. These changes in gene expression are not observed with bipolar disorder or non-psychotic major depression data sets, or in dentate neurons of rats treated chronically with clozapine. In addition, these changes in gene expression in schizophrenia are not associated with patient demographics including age, sex, brain weight, body weight, post-mortem interval, or drug history. Decreases in expression level between the normal and schizophrenic data sets are observed in large, overlapping clusters of genes that encode for protein turnover (i.e. proteasome subunits and ubiquitin), mitochondrial oxidative energy metabolism (i.e. isocitrate, lactate, malate, NADH and succinate dehydrogenases; cytochrome C oxidase and ATP synthase) and genes associated with neurite outgrowth, cytoskeletal proteins and synapse plasticity. These sets of genes are useful in that they provide the basis for the identification of novel therapeutic protein targets for the treatment of schizophrenia, potential diagnostic markers for schizophrenia, and markers for evaluating the therapeutic response to antipsychotic agents.

The invention therefore provides nucleic acids which can be used collectively in methods of the present invention, e.g. for diagnosing or treating schizopherenia, or for monitoring a therapy (for example, the administration of one or more drugs or other therapeutic compounds) to treat schizopherenia in an individual. Such collections of nucleic acids, are also referred here as a “gene signature” and comprise collections of nucleic acid sequences that are demonstrated (e.g., in the Examples of this application) to exhibit robust changes in gene expression in individuals with schzopherenia relative to control or reference groups who do not have or exhibit symptoms of that disease.

In one aspect, therefore, the invention provides methods in which a gene signature of the invention is used to diagnose schizophrenia in an individual. Such methods generally involve obtaining a cell or tissue sample from an individual who is either suspected of having schizopherenia or who is at risk for that disease (e.g., because of a family history of schizopherenia), and detecting or otherwise determining the expression level for at least one gene (i.e., one nucleic acid) in a gene signature of the invention. The determined expression level(s) for the one or more nucleic acids are then compared to expression levels of those nucleic acids in an individual (which can actually be the average from a collection of individuals) who does not have schizopherenia. A substantial or statistically significant difference in the expression level(s) of the nucleic acid in the first individual relative to the levels of expression in an individual(s) not having schizopherenia then indicates that the individual being tested does have, or is at risk of developing schizopherenia.

In another embodiment, the invention provides methods (e.g. screening methods) for identifying compounds that can be used to treat schizophrenia. Generally speaking, such methods involve contacting a cell or tissue sample with a test compound, determining the expression in the cell or tissue sample, of one or more nucleic acids in a gene signature of the invention. The expression level(s) thus determined can then be compared to expression level(s) for the nucleic acid(s) in a control cell or tissue sample that is not contacted with the test compound. In these methods, a difference in the expression of the nucleic acid(s) when the cell or tissue sample is contacted with the test compound indicates that the test compound can be used, or is at least a candidate compound, for treating schizoprenia. In preferred embodiments of those methods, a neural cell (or more precisely, a neural cell line) is used. However, other types of cells or tissue samples can also be used.

In still other embodiments, the invention provides methods for monitoring a therapy or a “therapeutic response” in an individual who is being treated for schizophrenia. Such methods generally involve steps of determining, e.g., in a cell or tissue sample from the individual, the level of expression for one or more genes in a gene signature of the invention, and comparing these determined expression levels to level(s) of expression, e.g., in a cell or tissue sample not having or undergoing a therapy for schizophrenia. More typically, expression levels are compared to a collective average of expression levels in individuals who do not have and/or are not undergoing therapy for schizophrenia. Alternatively, the determined expression levels can be compared to a collective average of expression levels in individuals who have successfully undergone therapy for schizophrenia. In such methods, a successful therepautic response is indicated if the determined expression level(s) is (are) similar to the corresponding expression level(s) in individuals against which the determined expression levels are compared.

In all of the above-described methods, the “gene signature” nucleic acids can be any one of, or a combination of two gene signature nucleic acids described here. Preferred nucleic acids are set forth in Table 14, infra, and in SEQ ID NOS: 1-249. In preferred embodiments, expression levels for a plurality of these gene signature nucleic acids are determined is used. For example, the expression levels for at least 10, 20, 30, 40, 50, 60, 70, 80, 90, 100, 110, 120, 130, 140, 150 or more gene signature nucleic acids can be determined and used in the various methods of this invention. In particularly preferred embodiments, expression levels are determined for at least 14, for at least 28, or for at least 42 gene signature nucleic acids.

Another aspect of the invention is a kit for diagnosing schizophrenia in an individual comprising a plurality of nucleic acid probes. In this aspect of the invention, each of the probes contained in the kit specifically hybridizes of any one or more of the genes identified in Table 14. In yet another aspect of the invention is a kit for diagnosing schizophrenia in an individual comprising a plurality of primer pairs. In this aspect of the invention, each of the primer pairs contained in the kit specifically amplifies any one or more of the genes identified in Table 14. In preferred embodiments, one or more polymerase are used to amplify the genes. Preferably, the kits of the present invention further comprise a detectable label.

In yet another embodiment, the diagnostic methods of the invention comprise a step of measuring the expression level of any one or more of the genes identified in Table 14, infra, in an individual who is undergoing treatment for schizophrenia. The one or more measured expression levels may then be compared to the expression levels of the corresponding gene signatures described herein for individuals who do not have schizophrenia. A therapeutic response is indicated if the expression levels in the individual who is undergoing treatment for schizophrenia are similar to the expression levels (gene signature) derived from tissue samples of individuals who do not have schizophrenia.

### BRIEF DESCRIPTION OF THE DRAWINGS

FIGS. 1A-D is a representative photomicrographs of dentate granule neurons collected from human hippocampus. FIG. 1A depicts low (12.5X) magnification of the Nissl-stained section. FIG. 1B depicts high (40X) magnification of Nissl-stained section. FIG. 1C depicts high (40X) magnification of the tissue surrounding the dentate cell layer within the transfer film. FIG. 1D depicts high (40X) magnification of the dentate neurons embedded within the transfer film.

FIGS. 2A-C show scatter plots as follows: FIG. 2A shows scatter plots of gene expression changes in the dentate gyrus for schizophrenia of cohort 1 (n=8-10 per group); FIG. 2B shows scatter plots of gene expression changes in the dentate gyrus for bipolar disorder of cohort 1 (n=8-10 per group); and FIG. 2C shows scatter plots of gene expression changes in the dentate gyrus for depression cases of cohort 1 (n=8-10 per group). For each gene (black dot), the ratio of the average relative expression value in disease versus that for controls (n=9) is plotted on the y-axis. The x-axis is the average intensity for the specified gene in control cases.

FIGS. 3A-B shows the numbers of modulated genes identified in cohorts 1 and 2 as follows: FIG. 3A shows the number of decreased genes identified in cohorts 1 and 2 to the left or right of each circle, respectively (p<0.05;>25% decrease). FIG. 3B shows the number of increased genes identified in cohorts 1 and 2 to the left or right of each circle, respectively (p<0.5;>25% decrease). The maximum percent overlap between adjacent circles (% overlap) is presented. The 36% and 28% overlaps in decreases and increases between the two cohorts were 7- and 5-fold more likely than would be expected by chance, respectively, from the 12,388 and 12,725 genes whose basal expression were detected in either cohort.

FIGS. 4A-F show the two-way ANOVA of the 263 genes that showed co-directional changes in both schizophrenia cohorts. Distribution of the number of genes (“count”) whose variance changed as a function of each demographic factor, plotted for several significance value ranges (“p value”). FIG. 4A is a plot of the significance value range versus the distribution for Disease. FIG. 4B is a plot of the of the significance value range versus the distribution for Brain pH. FIG. 4C is a plot of the significance value range versus the distribution for Brain Weight. FIG. 4D is a plot of the significance value range versus the distribution for PMI. FIG. 4E is a plot of the significance value range versus the distribution for Brain pH. FIG. 4F is a plot of the significance value range versus the distribution for Sex. Note the expanded scale for p values between 0 and 0.2 for Disease (FIG. 4A) and Brain pH (4 C).

FIGS. 5A-D show expression of four genes representative in the individual controls and schizophrenic cases as follows: FIG. 5A shows expression of the proteasome; FIG. 5B shows expression of ubiquitin, FIG. 5C shows the expression of lactate dehydrogenase A and FIG. 5D shows the expression of NADH dehydrogenase. Mean expression level for each group is indicated by the black bar. Arrows identify the 3 patients who were not on antipsychotics at the time of death. These genes were also not altered in dentate neurons of rats (n=10/group) treated with the pharmacologically complex antipsychotic drug, clozapine, used by half of the schizophrenic patients. Expression levels for the half of the schizophrenia cases who had been treated with clozapine were evenly distributed within the entire group.

FIG. 6 is a schematic summarizing the biochemical pathways for which the most affected mRNA species were found in schizophrenia, and their relation to excitatory neurotransmitter inputs, pH control, and synaptic functions.

### BRIEF DESCRIPTION OF THE TABLES

Table 1. Lists genes relevant to mitochondria that were identified as being significantly altered in schizophrenia relative to normal controls (n=10-13/group). The average change (“ratio”) and statistical relevance (“P value”) is presented for the hippocampal dentate gyrus neurons (“Dentate”) and cells collected in the CA3 region of the hippocampus (“CA3”).

Table 2. Lists genes relevant to non-mitochondrial energy metabolism that were identified as significantly altered in schizophrenia relative to normal controls (n=10-13/group). The average change (“ratio”) and statistical relevance (“P value”) is presented for the hippocampal dentate gyrus neurons (“Dentate”) and cells collected in the CA3 region of the hippocampus (“CA3”).

Table 3. Lists genes relevant to the ubiquitin-proteasome system that were identified as significantly altered in schizophrenia relative to normal controls (n=10-13/group). The average change (“ratio”) and statistical relevance (“P value”) is presented for the hippocampal dentate gyrus neurons (“Dentate”) and cells collected in the CA3 region of the hippocampus (“CA3”).

Table 4. Lists lysosomal genes that were identified as significantly altered in schizophrenia relative to normal controls (n=10-13/group). The average change (“ratio”) and statistical relevance (“P value”) is presented for the hippocampal dentate gyrus neurons (“Dentate”) and cells collected in the CA3 region of the hippocampus (“CA3”).

Table 5. Lists genes relevant to immune/inflammatory mediators that were identified as significantly altered in schizophrenia relative to normal controls (n=10-13/group). The average change (“ratio”) and statistical relevance (“P value”) is presented for the hippocampal dentate gyrus neurons (“Dentate”) and cells collected in the CA3 region of the hippocampus (“CA3”).

Table 6. Lists genes relevant to synaptic plasticity, growth and development that were identified as significantly altered in schizophrenia relative to normal controls (n=10-13/group). The average change (“ratio”) and statistical relevance (“P value”) is presented for the hippocampal dentate gyrus neurons (“Dentate”) and cells collected in the CA3 region of the hippocampus (“CA3”).

Table 7. List binomial probabilities for some gene groups in which disproportionately high levels of individual genes are down regulated by schizophrenia in dentate.

Table 8. Lists the diagnostic category (Description), case ID number, case age, sex, PMI, brain pH, brain weight, body weight, and cumulative antipsychotic exposure of the 65 cases in Cohorts 1 and 2.

Table 9. Lists groupings of altered genes into functional pathways based upon binomial probability computation or Fisher exact test calculated by the EASE software. The functional categories in parentheses are for the EASE calculations. Bonferroni corrections (Bonf.) are a division of the p value score by the 11,000 distinct terms in gene ontology for the Binomial method and 9,000 terms used in EASE. A value of 1 indicated non-significant p value. Unmarked boxes represent terms not used by EASE or our binomial analysis.

Table 10. Lists genes relevant to the mitochondria and energy metabolism system that were identified as significantly altered in schizophrenia relative to normal controls (n=10-13/group). The average change (“ratio”) and statistical relevance (“P value”) is presented for the hippocampal dentate gyrus neurons in Cohorts 1 and 2.

Table 11. Lists genes relevant to the ubiquitin-proteasome system that were identified as significantly altered in schizophrenia relative to normal controls (n=10-13/group). The average change (“ratio”) and statistical relevance (“P value”) is presented for the hippocampal dentate gyrus neurons in Cohorts 1 and 2.

Table 12. Lists genes relevant to neuronal plasticity, growth and development that were identified as significantly altered in schizophrenia relative to normal controls (n=10-13/group). The average change (“ratio”) and statistical relevance (“P value”) is presented for the hippocampal dentate gyrus neurons in Cohorts 1 and 2.

Table 13. Validation of representative changes in mitochondrial, proteasome, ubitquitin, and neuronal plasticity genes using TawMan Q-PCR. Microarray Cohort 1: n=9/group, Cohort 2:n=14-15/group Q-PCR Cohorts 1 and 2: n=22 control, 20 schizophrenic cases.

Table 14. List of genes that were identified as significantly altered in schizophrenia relative to normal controls (n=10-13/group). The average change (“ratio”) and statistical relevance (“P value”) is presented for the hippocampal dentate gyrus neurons in Cohorts 1 and 2.

### DETAILED DESCRIPTION OF THE INVENTION

The present invention is now described, in detail, by way of the following particular examples. However, the use of such examples is illustrative only and in no way limits the scope or meaning of this invention or any exemplified term. Nor is the invention limited to any preferred embodiments(s) described herein. Indeed, many modifications and variations of the invention will be apparent to those skilled in the art upon reading this specification, and such “equivalents” can be made without departing from the invention in spirit or scope.

### EXAMPLE 1

### Identification of Mitochondrial; Non-Mitochondrial Energy; Ubiquitin Proteasome; Lysosomal; Immune/Inflammatory Mediator; and Synaptic Plasticity, Growth and Development Genes Differentially Expressed in Schizophrenia

LCM and cDNA microarrays were used to profile gene expression within hippocampal dentate granule or CA3 neurons in normal controls and in patients with schizophrenia, bipolar disorder, or depression. Reported is the specific down-regulation of large numbers of genes in the hippocampus of schizophrenic patients that encode for a few distinct macromolecular complexes. These complexes are involved in mitochondrial function, energy metabolism, proteasome function, lysosomal function, and synaptic transmission.

### Materials and Methods

Human tissue: All post-mortem brain tissues used in the present study were obtained from the Stanley Foundation Neuropathology Consortium. The patients were diagnosed according to DSM-IV criteria and comprised those with schizophrenia, bipolar disease, depression, and also included control patients who were free of diagnosed psychiatric disease (n=10-13 patients per group).
Preparation of sections: Ten μm-thick frozen coronal sections that contained the hippocampus were thaw-mounted onto 2×3 inch gelatin-coated microscope slides and stored at −80 deg C. until use.
Cell capture: Each section was quick-thawed, fixed in 75% ethanol, re-hydrated in dH2O and stained for 2 min. with Arcturus Histogene™ staining solution. The sections were dehydrated in ascending ethanols, placed into xylene for 5 minutes and air-dried for 15 minutes prior to laser-capture. Approximately 1000 dentate granule cells or CA3 cells were micro-dissected from each of 2-3 sections using an Arcturus PixCell II-eTM laser-capture microscope. All tissue collection and subsequent procedures were conducted in a blind and counterbalanced manner between the four patient groups.
RNA Amplification: The total RNA extracted from the dentate granule or CA3 cells of each patient sample underwent two rounds of linear amplification using the Arcturus RiboAmp kit, yielding an average of 167 ug of amplified RNA (aRNA) for each sample. Equal amounts of each control sample were pooled to generate a common reference sample, against which the individual samples were hybridized on microarrays.
RNA Labeling for Agilent Microarrays. 400 ng of aRNA (individual or common reference sample) was mixed with 400 ng of random hexamers (Promega) in a volume of 50 ul, denatured for 10 min at 700° C., chilled on ice, and collected by brief centrifugation. 50 ul of a 2× master mix (containing First Strand Reaction Buffer, DTT, dNTPs and MMLV-RT from the Agilent Direct-Label cDNA Synthesis Kit and 2.5 ul of 1.0 uM Cyanine 3-dCTP or Cyanine 5-dCTP from Perkin-Elmer NEN) was added on ice. Reactions were incubated for 10 min at 25° C., 1 h at 42° C., and 10 min at 70° C., chilled on ice, and collected by brief centrifugation. Following treatment with 2 ul of 0.05 mg/ml RNase IA (Agilent Technologies) for 30 min at room temperature, the labeled cDNA was purified using the QIAquick PCR Purification Kit (Qiagen) following the manufacturer's directions, with an additional wash step of 0.75 ml 35% guanidine hydrochloride prior to washing with Qiagen buffer PE. The purified Cy3 and Cy5-labeled cDNAs were combined, concentrated to dryness in a Speedvac centrifuge concentrator (Savant), and resuspended in 7.5 ul H2O.
Hybridization, Washing, and Scanning of Agilent Human 1 cDNA Microarrays. 2.5 ul Deposition Control Target (Operon), 2.5 ul human 1 mg/ml COT-1 DNA (Invitrogen), and 12.5 ul 2× Hybridization Buffer (Agilent) was added to the labeled cDNA. The mixture was heated at 98° C. for 2 min, centrifuged for 5 min at room temperature and applied to coverslipped Agilent Human 1 cDNA Microarrays. Arrays were hybridized for 17 hr at 65° C. in humidified chambers (Corning). Coverslips were removed by submerging briefly in 0.5×SSC, 0.01% SDS, then arrays were agitated for 5 min at room temperature in the same buffer, followed by 2 min in room temperature 0.06×SSC. Slides were dried by centrifugation at 500×g and scanned using the Agilent G2565AA Microarray Scanner System.
Microarray Data Analysis: Only those genes that produced an average intensity of at least 300 in at least one of the sample groups were evaluated. The log ratio for each sample/reference value was determined for each gene and the mean log ratio calculated for each patient group. Log ratios are utilized in the processing of two-channel array data because it is expected that the distribution of log ratios is closer to normality than the distribution of ratios. For each gene, the mean ratio for the patient group was then divided by the mean ratio of the control group to calculate the fold change between the two groups. Welch t test p values were determined by comparing schizophrenia/reference log ratios to control/reference log ratios for each gene. Genes were selected as those with a p value<0.05 and a fold change compared to controls of greater than 25%.

### Results

Tables 1-6 below provide lists of genes identified as being significantly altered in schizophrenia relative to normal controls (n=10-13/group). These genes were in each table according to their relevance to mitochondria (Table 1), non-mitochondrial energy metabolism (Table 2), the ubiquitin-proteasome system (Table 3), lysosome (Table 4), immune/inflammatory mediators (Table 5), and synaptic plasticity, growth and development (Table 6). In each table, the average change (“ratio”) and statistical relevance (“P value”) is presented for the hippocampal dentate gyrus (“Dentate”) and cells collected in the CA3 region of the hippocampus (“CA3”).

Microarray experiments, such as the ones described here, simultaneously measure changes in the expression of many different genes. Therefore, there is some concern that many of the observed changes may result from chance fluctuations and are not representative of a real disease effect on gene expression. The likelihood of chance fluctuations is significantly less is multiple changes are observed among genes of a common pathway, macromolecular complex or other biological functional group. This is because, where such a “cluster” of genes is truly affected by a disease, the proportion of gene changes within that cluster will be significantly greater than the proportion of gene changes among all genes expressed by the cell(s).

In the experiments described here, binomial probabilities are used to assess whether the proportion of genes in a functional group that are declared “hits” (based on the cut-off criteria for p-values and ratios) is significantly greater than the average proportion of hits among all genes. For example, in the dentate experiments described here, 9342 genes were expressed at levels that pass the abundance cut-off requirement of 300. Of these expressed genes, a total of 576 genes were downregulated in schizophrenia with p-values below 0.05 and ratios less than 0.8. Hence, the probability that any randomly selected gene is downregulated is schizophrenia is 576/9342 or 6.17%. Of the expressed genes, 55 are in the proteasome pathway and 14 (i.e., approximately 25%) of those genes are down-regulated with p-values and ratios that fall below the above-mentioned cut-off values. Yet the probability that 14 randomly selected genes (out of the total 9342 genes expressed) are all down regulated is only (0.0617)¹⁴=4.5×10⁻⁶. Similar binomial probabilities are set forth in Table 7, infa, for other pathway groups. Such a low probabilities give great confidence that the fluctuations observed among the different pathway genes are real effects of the schizophrenia disease and not merely a random fluctuation in gene expression.

TABLE 1

| | | | | CA3 | |
|---|---|---|---|---|---|
| | | Dentate | | | P |
| Gene Description | Genbank | Ratio | P Value | Ratio | Value |
| 2,4-dienoyl CoA reductase 1, mitochondrial | L26050 | | | 0.726 | 0.0377 |
| 3-hydroxybutyrate dehydrogenase (heart, | AW246790 | | | 0.687 | 0.0067 |
| mitochondrial) | | | | | |
| acetyl-Coenzyme A acetyltransferase 1 | D90228 | 0.799 | 0.00793 | | |
| (acetoacetyl Coenzyme A thiolase) | | | | | |
| acetyl-Coenzyme A acyltransferase 2 | D16294 | 0.733 | 0.01538 | | |
| (mitochondrial 3-oxoacyl-Coenzyme A thiolase) | | | | | |
| acyl-Coenzyme A dehydrogenase, C-4 to C-12 | AA505399 | | | 0.751 | 0.0459 |
| straight chain | | | | | |
| AFG3 ATPase family gene 3-like 2 (yeast) | Y18314 | 0.673 | 0.00791 | | |
| alternative; H. sapiens gene for phosphate | X77337 | 0.756 | 0.00106 | | |
| carrier. | | | | | |
| arginase, type II | D86724 | 0.777 | 0.02395 | | |
| ATP synthase, H+ transporting, mitochondrial | X60221 | 0.649 | 0.00126 | | |
| F0 complex, subunit b, isoform 1 | | | | | |
| ATP synthase, H+ transporting, mitochondrial | BE383477 | 0.646 | 0.00084 | 0.621 | 0.0328 |
| F0 complex, subunit c (subunit 9) isoform 3 | | | | | |
| ATP synthase, H+ transporting, mitochondrial | D13119 | 0.795 | 0.01715 | | |
| F0 complex, subunit c (subunit 9), isoform 2 | | | | | |
| ATP synthase, H+ transporting, mitochondrial | NM_007100 | 0.787 | 0.00134 | | |
| F0 complex, subunit e | | | | | |
| ATP synthase, H+ transporting, mitochondrial | AI138629 | 0.755 | 0.00252 | | |
| F0 complex, subunit g | | | | | |
| ATP synthase, H+ transporting, mitochondrial | D14710 | 0.682 | 0.00078 | 0.680 | 0.0375 |
| F1 complex, alpha subunit, isoform 1, cardiac | | | | | |
| muscle | | | | | |
| ATP synthase, H+ transporting, mitochondrial | AF052955 | 0.781 | 0.00427 | | |
| F1 complex, epsilon subunit | | | | | |
| ATP synthase, H+ transporting, mitochondrial | D16563 | 0.697 | 0.00483 | 0.590 | 0.0491 |
| F1 complex, gamma polypeptide 1 | | | | | |
| ATP synthase, H+ transporting, mitochondrial | AI215675 | 0.679 | 0.00008 | 0.665 | 0.0455 |
| F1 complex, O subunit (oligomycin sensitivity | | | | | |
| conferring protein) | | | | | |
| ATP/ADP translocator; Human heart/skeletal | J04982 | 0.652 | 0.00035 | | |
| muscle ATP/ADP translocator (ANT1) gene, | | | | | |
| complete cds. | | | | | |
| cytochrome b-245, beta polypeptide (chronic | X04011 | 0.747 | 0.00029 | 0.775 | 0.0365 |
| granulomatous disease) | | | | | |
| cytochrome b5 outer mitochondrial membrane | AB009282 | 0.757 | 0.00878 | 0.671 | 0.0142 |
| precursor | | | | | |
| cytochrome c oxidase subunit IV isoform 1 | NM_001861 | 0.786 | 0.00198 | | |
| cytochrome c oxidase subunit VIc | X13238 | 0.738 | 0.00244 | | |
| cytochrome c oxidase subunit VIIa polypeptide | AA525082 | 0.660 | 0.00026 | | |
| 2 (liver) | | | | | |
| cytochrome c oxidase subunit VIIa polypeptide | AI355189 | 0.776 | 0.00910 | | |
| 2 like | | | | | |
| cytochrome c oxidase subunit VIIb | AI209213 | 0.654 | 0.00085 | 0.771 | 0.0399 |
| cytochrome c; Human somatic cytochrome c | M22877 | 0.632 | 0.00205 | 0.576 | 0.0311 |
| (HCS) gene, complete cds. | | | | | |
| cytochrome P450 4F4 | AAC52358 | 0.772 | 0.03089 | | |
| cytochrome P450IIE1; Human cytochrome | J02843 | | | 0.769 | 0.0377 |
| P450IIE1 (ethanol-inducible) gene, complete | | | | | |
| cds. | | | | | |
| diaphorase (NADH) (cytochrome b-5 | M16462 | 0.795 | 0.01151 | | |
| reductase) | | | | | |
| diazepam binding inhibitor (GABA receptor | AA868701 | 0.738 | 0.00040 | | |
| modulator, acyl-Coenzyme A binding protein) | | | | | |
| dihydrolipoamide dehydrogenase (E3 | J03620 | 0.779 | 0.00246 | 0.728 | 0.0162 |
| component of pyruvate dehydrogenase | | | | | |
| complex, 2-oxo-glutarate complex, branched | | | | | |
| chain keto acid dehydrogenase complex) | | | | | |
| electron-transfer-flavoprotein, alpha | W19485 | 0.698 | 0.00087 | | |
| polypeptide (glutaric aciduria II) | | | | | |
| electron-transfer-flavoprotein, beta polypeptide | X71129 | 0.787 | 0.03428 | | |
| enoyl Coenzyme A hydratase 1, peroxisomal | AI718453 | | | 0.780 | 0.0316 |
| EST, Highly similar to CY1_HUMAN | AK026633 | 0.712 | 0.00815 | 0.649 | 0.0194 |
| Cytochrome c1, heme protein, mitochondrial | | | | | |
| precursor [H. sapiens] | | | | | |
| EST, Moderately similar to NUML_HUMAN | AL110150 | 0.669 | 0.01789 | 0.593 | 0.0284 |
| NADH-ubiquinone oxidoreductase MLRQ | | | | | |
| subunit (Complex I-MLRQ) (CI-MLRQ) | | | | | |
| [H. sapiens] | | | | | |
| EST, Weakly similar to TTC1_HUMAN | AK000594 | 0.781 | 0.03182 | | |
| Tetratricopeptide repeat protein 1 (TPR repeat | | | | | |
| protein 1) [H. sapiens] | | | | | |
| fatty-acid-Coenzyme A ligase, very long-chain 1 | NM_003645 | 0.763 | 0.00873 | | |
| glutamic-oxaloacetic transaminase 2, | M22632 | 0.767 | 0.01843 | 0.735 | 0.0367 |
| mitochondrial (aspartate aminotransferase 2) | | | | | |
| glycine C-acetyltransferase (2-amino-3- | AF077740 | | | 0.773 | 0.0409 |
| ketobutyrate coenzyme A ligase) | | | | | |
| glycine cleavage system protein H | D00723 | 0.674 | 0.01163 | | |
| (aminomethyl carrier) | | | | | |
| H. sapiens gene encoding enoyl-CoA | X98126 | 0.787 | 0.00036 | | |
| hydratase, exon 1(and joined CDS). | | | | | |
| H. sapiens gene for mitochondrial ATP | X69907 | 0.753 | 0.00152 | 0.668 | 0.0154 |
| synthase c subunit (P1 form). | | | | | |
| H. sapiens gene for mitochondrial ATP | X69908 | 0.798 | 0.00030 | | |
| synthase c subunit (P2 form). | | | | | |
| Homo sapiens (clone f17252) ubiquinol | L32977 | 0.587 | 0.00007 | | |
| cytochrome c reductase Rieske iron-sulphur | | | | | |
| protein (UQCRFS1) gene, exon 2. | | | | | |
| Homo sapiens ATP synthase beta subunit | M27132 | 0.775 | 0.00423 | | |
| precursor (ATPSB) gene, complete cds. | | | | | |
| Homo sapiens cDNA: FLJ22657 fis, clone | AK026310 | 0.718 | 0.00034 | | |
| HS107791, highly similar to HUMCYB5 Human | | | | | |
| cytochrome b5 mRNA. | | | | | |
| Homo sapiens cDNA: FLJ22970 fis, clone | AK026623 | | | 0.696 | 0.0496 |
| KAT10766, highly similar to HUMCOXNE | | | | | |
| Homo sapiens nuclear-encoded mitochondrial | | | | | |
| cytochrome c oxidase Va subunit mRNA. | | | | | |
| Human cytochrome c oxidase subunit VIa | U83702 | 0.722 | 0.00060 | | |
| gene, exon 3 and complete cds. | | | | | |
| Human cytochrome c oxidase subunit VIII | J04823 | 0.746 | 0.00150 | 0.704 | 0.0105 |
| (COX8) mRNA, complete cds. | | | | | |
| Human DNA sequence from BAC 15E1 on | AL021546 | 0.742 | 0.00142 | | |
| chromosome 12. Contains Cytochrome C | | | | | |
| Oxidase Polypeptide VIa-liver precursor gene, | | | | | |
| 60S ribosomal protein L31 pseudogene, pre- | | | | | |
| mRNA splicing factor SRp30c gene, two | | | | | |
| putative genes, ESTs, STSs and putative CpG | | | | | |
| islands | | | | | |
| Human gene for ATP synthase alpha subunit, | D28126 | 0.717 | 0.00768 | 0.661 | 0.0361 |
| complete cds (exon 1 to 12). | | | | | |
| inner membrane protein, mitochondrial | L42572 | 0.780 | 0.00339 | 0.637 | 0.0485 |
| (mitofilin) | | | | | |
| isocitrate dehydrogenase 2 (NADP+), | X69433 | 0.782 | 0.00005 | | |
| mitochondrial | | | | | |
| isocitrate dehydrogenase 3 (NAD+) alpha | U07681 | 0.705 | 0.00451 | | |
| isocitrate dehydrogenase 3 (NAD+) beta | BE409783 | 0.740 | 0.00356 | 0.685 | 0.0456 |
| L-3-hydroxyacyl-Coenzyme A dehydrogenase, | X96752 | 0.762 | 0.03391 | 0.659 | 0.0337 |
| short chain | | | | | |
| liver isoform; Homo sapiens cytochrome-c | AF134406 | 0.656 | 0.00051 | | |
| oxidase subunit VIIaL precursor (COX7AL) | | | | | |
| gene, complete cds. | | | | | |
| low molecular mass ubiquinone-binding protein | AL036415 | 0.654 | 0.00011 | | |
| (9.5 kD) | | | | | |
| malate dehydrogenase 1, NAD (soluble) | D55654 | 0.695 | 0.01172 | | |
| malate dehydrogenase 2, NAD (mitochondrial) | AW249275 | 0.609 | 0.00002 | 0.621 | 0.0049 |
| malic enzyme 3, NADP(+)-dependent, | X79440 | | | 0.665 | 0.0433 |
| mitochondrial | | | | | |
| metaxin 2 | AF053551 | 0.681 | 0.00044 | 0.664 | 0.0488 |
| mitochondrial carrier homolog 1 | AF176006 | 0.723 | 0.00039 | 0.685 | 0.0157 |
| mitochondrial ribosomal protein L3 | NM_007208 | 0.708 | 0.00202 | | |
| mitochondrial ribosomal protein L32 | AF161401 | | | 0.743 | 0.0159 |
| mitochondrial ribosomal protein L33 | NM_004891 | 0.691 | 0.00001 | | |
| mitochondrial ribosomal protein S18A | AK001410 | 0.786 | 0.03928 | | |
| mitochondrial ribosomal protein S30 | AL355715 | | | 0.673 | 0.0186 |
| NADH dehydrogenase (ubiquinone) 1 alpha | AF087661 | 0.789 | 0.00275 | 0.715 | 0.0099 |
| subcomplex, 10, 42 kDa | | | | | |
| NADH dehydrogenase (ubiquinone) 1 alpha | NM_002490 | | | 0.690 | 0.0364 |
| subcomplex, 6, 14 kDa | | | | | |
| NADH dehydrogenase (ubiquinone) 1 beta | AF047181 | 0.592 | 0.00013 | 0.681 | 0.0265 |
| subcomplex, 5 (16 kD, SGDH) | | | | | |
| NADH dehydrogenase (ubiquinone) 1 beta | AF035840 | 0.706 | 0.00532 | | |
| subcomplex, 6 (17 kD, B17) | | | | | |
| NADH dehydrogenase (ubiquinone) 1 beta | AF112200 | | | 0.787 | 0.0368 |
| subcomplex, 7, 18 kDa | | | | | |
| NADH dehydrogenase (ubiquinone) 1, | BE266480 | | | 0.675 | 0.0247 |
| alpha/beta subcomplex, 1, 8 kDa | | | | | |
| NADH dehydrogenase (ubiquinone) 1, | AF047184 | 0.794 | 0.00186 | 0.730 | 0.0460 |
| subcomplex unknown, 1 (6 kD, KFYI) | | | | | |
| NADH dehydrogenase (ubiquinone) Fe—S | AF050640 | 0.798 | 0.00938 | | |
| protein 2, 49 kDa (NADH-coenzyme Q | | | | | |
| reductase) | | | | | |
| NADH dehydrogenase (ubiquinone) Fe—S | AF020351 | 0.588 | 0.00010 | 0.635 | 0.0202 |
| protein 4 (18 kD) (NADH-coenzyme Q | | | | | |
| reductase | | | | | |
| NADH dehydrogenase (ubiquinone) Fe—S | AF038406 | 0.755 | 0.01283 | 0.785 | 0.0455 |
| protein 8 (23 kD) (NADH-coenzyme Q | | | | | |
| reductase) | | | | | |
| NADH dehydrogenase (ubiquinone) | AW250734 | | | 0.696 | 0.0090 |
| flavoprotein 1, 51 kDa | | | | | |
| ornithine aminotransferase (gyrate atrophy) | M12267 | 0.752 | 0.01470 | | |
| phosphogluconate dehydrogenase | U30255 | 0.754 | 0.00104 | 0.778 | 0.0429 |
| precursor; Human mitochondrial creatine | J04469 | | | 0.696 | 0.0171 |
| kinase (CKMT) gene, complete cds. | | | | | |
| programmed cell death 8 (apoptosis-inducing | AF100928 | 0.794 | 0.01132 | | |
| factor) | | | | | |
| propionyl Coenzyme A carboxylase, alpha | S79219 | 0.752 | 0.00090 | | |
| polypeptide | | | | | |
| pyruvate dehydrogenase (lipoamide) beta | NM_000925 | 0.751 | 0.03512 | | |
| Pyruvate dehydrogenase complex, lipoyl- | U82328 | 0.692 | 0.00460 | 0.555 | 0.0287 |
| containing component X; E3-binding protein | | | | | |
| SCO cytochrome oxidase deficient homolog 1 | AI332708 | 0.612 | 0.00034 | 0.624 | 0.0437 |
| (yeast) | | | | | |
| serine hydroxymethyltransferase 2 | NM_005412 | | | 0.706 | 0.0187 |
| (mitochondrial) | | | | | |
| similar to CI-AGGG; Homo sapiens NADH- | AF067166 | 0.704 | 0.00013 | | |
| ubiquinone oxidoreductase AGGG subunit | | | | | |
| precursor homolog mRNA, nuclear gene | | | | | |
| encoding mitochondrial protein, complete cds. | | | | | |
| solute carrier family 25 (mitochondrial carrier; | J02683 | 0.621 | 0.00029 | 0.694 | 0.0220 |
| adenine nucleotide translocator), member 5 | | | | | |
| succinate dehydrogenase complex, subunit A, | L21936 | 0.739 | 0.00478 | 0.662 | 0.0396 |
| flavoprotein (Fp) | | | | | |
| succinate dehydrogenase complex, subunit B, | AW960231 | 0.660 | 0.00002 | 0.704 | 0.0230 |
| iron sulfur (Ip) | | | | | |
| succinate dehydrogenase complex, subunit C, | D49737 | 0.775 | 0.00662 | | |
| integral membrane protein, 15 kD | | | | | |
| succinate dehydrogenase complex, subunit D, | NM_003002 | 0.660 | 0.00171 | | |
| integral membrane protein | | | | | |
| surfeit 1 | Z35093 | 0.759 | 0.00060 | | |
| thioredoxin reductase 1 | D88687 | 0.754 | 0.00343 | | |
| translocase of inner mitochondrial membrane | AW247564 | 0.625 | 0.00021 | 0.641 | 0.0325 |
| 17 homolog A (yeast) | | | | | |
| ubiquinol-cytochrome c reductase (6.4 kD) | AW163002 | 0.673 | 0.00011 | 0.745 | 0.0157 |
| subunit | | | | | |
| ubiquinol-cytochrome c reductase core protein I | AI373152 | 0.747 | 0.01916 | 0.718 | 0.0241 |
| ubiquinol-cytochrome c reductase core protein | NM_003366 | 0.784 | 0.00901 | 0.661 | 0.0261 |
| II | | | | | |
| ubiquinol-cytochrome c reductase hinge | AI093521 | 0.642 | 0.00069 | 0.621 | 0.0324 |
| protein | | | | | |
| voltage-dependent anion channel 1 | L06132 | 0.710 | 0.00278 | | |

TABLE 2

| | | Dentate | | CA3 | |
|---|---|---|---|---|---|
| Gene Description | Genbank | Ratio | P Value | Ratio | P Value |
| aldehyde dehydrogenase 9 family, | U34252 | 0.7402 | 0.00523 | | |
| member A1 | | | | | |
| ferrochelatase (protoporphyria) | D00726 | 0.7211 | 0.04746 | 0.706 | 0.0224 |
| H. sapiens gene for | X83464 | 0.7795 | 0.00007 | | |
| glucosephosphate isomerase (exon | | | | | |
| 15, 16, 17 and 18). | | | | | |
| H. sapiens lactate dehydrogenase B | X13800 | 0.7890 | 0.00152 | | |
| gene exon 8 (EC 1.1.1.27). | | | | | |
| Homo sapiens aldose reductase | AF032455 | 0.6683 | 0.00009 | 0.701 | 0.0105 |
| gene, complete cds. | | | | | |
| Homo sapiens COX17 (COX17) | AF269245 | | | 0.664 | 0.0289 |
| gene, exon 3. | | | | | |
| Homo sapiens gene for insulin | AB000732 | 0.7874 | 0.00831 | | |
| receptor substrate-2, complete cds. | | | | | |
| Homo sapiens insulin induced | U96876 | 0.6265 | 0.00159 | | |
| protein 1 (INSIG1) gene, complete | | | | | |
| cds. | | | | | |
| Human aldose reductase (AR) | M59783 | 0.6571 | 0.00019 | 0.733 | 0.0150 |
| gene, segment 2. | | | | | |
| Human glucose transporter 2 | L09674 | | | 0.709 | 0.0180 |
| (GLUT2) gene, exon 1. | | | | | |
| lactate dehydrogenase A | X02152 | 0.5732 | 0.00017 | 0.558 | 0.0211 |
| lactate dehydrogenase B | Y00711 | 0.6206 | 0.00009 | | |
| phosphofructokinase, muscle | M26066 | 0.6892 | 0.03342 | | |
| phosphorylase kinase, beta | X84908 | 0.7328 | 0.01187 | | |
| protein phosphatase 1, regulatory | NM_006241 | 0.7884 | 0.00771 | 0.590 | 0.0482 |
| (inhibitor) subunit 2 | | | | | |
| sialyltransferase 8A (alpha-N- | NM_003034 | 0.7236 | 0.02322 | | |
| acetylneuraminate: alpha-2,8- | | | | | |
| sialytransferase, GD3 synthase) | | | | | |

TABLE 3

| | | Dentate | | CA3 | |
|---|---|---|---|---|---|
| Gene Description | Genbank | Ratio | P Value | Ratio | P Value |
| 26S proteasome-associated pad1 homolog | U86782 | 0.717 | 0.00645 | 0.665 | 0.0484 |
| F-box and leucine-rich repeat protein 2 | AF176518 | 0.784 | 0.00313 | | |
| Homo sapiens cONA FLJ13228 fis, clone | AK023290 | | | 0.650 | 0.0414 |
| OVARC1000085, highly similar to Human | | | | | |
| mRNA for proteasome subunit HC5. | | | | | |
| Homo sapiens UbcM2 mRNA, complete cds. | AF085362 | 0.756 | 0.00076 | | |
| Homo sapiens ubiquitin carboxy-terminal | AF076269 | 0.640 | 0.00041 | | |
| hydrolase L1 (UCHL1) gene, exon 3. | | | | | |
| Homo sapiens ubiquitin gene. | X04803 | 0.578 | 0.00002 | 0.631 | 0.0195 |
| Human mannosidase, beta A, lysosomal | AF224669 | 0.774 | 0.00026 | 0.791 | 0.0278 |
| (MANBA) gene, and ubiquitin-conjugating | | | | | |
| enzyme E2D 3 (UBE2D3) genes, complete | | | | | |
| cds. | | | | | |
| proteasome (prosome, macropain) 26S | L02426 | 0.778 | 0.00163 | | |
| subunit, ATPase, 1 | | | | | |
| proteasome (prosome, macropain) 26S | BE397250 | | | 0.653 | 0.0284 |
| subunit, ATPase, 4 | | | | | |
| proteasome (prosome, macropain) 26S | AF006305 | 0.725 | 0.00134 | | |
| subunit, ATPase, 6 | | | | | |
| proteasome (prosome, macropain) 26S | D44466 | | | 0.737 | 0.0428 |
| subunit, non-ATPase, 1 | | | | | |
| proteasome (prosome, macropain) 26S | AB009619 | | | 0.686 | 0.0200 |
| subunit, non-ATPase, 10 | | | | | |
| proteasome (prosome, macropain) 26S | D38047 | 0.633 | 0.00007 | 0.618 | 0.0152 |
| subunit, non-ATPase, 8 | | | | | |
| proteasome (prosome, macropain) 26S | AB003177 | 0.745 | 0.00031 | | |
| subunit, non-ATPase, 9 | | | | | |
| proteasome (prosome, macropain) activator | AA310524 | 0.728 | 0.01419 | 0.716 | 0.0167 |
| subunit 1 (PA28 alpha) | | | | | |
| proteasome (prosome, macropain) inhibitor | D88378 | 0.750 | 0.00456 | | |
| subunit 1 (PI31) | | | | | |
| proteasome (prosome, macropain) subunit, | AI889267 | 0.679 | 0.00049 | | |
| alpha type, 1 | | | | | |
| proteasome (prosome, macropain) subunit, | D00760 | 0.735 | 0.02299 | 0.619 | 0.0429 |
| alpha type, 2 | | | | | |
| proteasome (prosome, macropain) subunit, | AF054185 | 0.799 | 0.03398 | | |
| alpha type, 7 | | | | | |
| proteasome (prosome, macropain) subunit, | AL031259 | 0.713 | 0.00080 | 0.677 | 0.0357 |
| beta type, 1 | | | | | |
| proteasome (prosome, macropain) subunit, | D26598 | 0.705 | 0.01348 | 0.647 | 0.0275 |
| beta type, 3 | | | | | |
| proteasome (prosome, macropain) subunit, | D29012 | 0.660 | 0.00007 | 0.662 | 0.0079 |
| beta type, 6 | | | | | |
| ubiquitin A-52 residue ribosomal protein | AF075321 | 0.798 | 0.04430 | | |
| fusion product 1 | | | | | |
| ubiquitin B | BE250544 | 0.638 | 0.00001 | 0.680 | 0.0150 |
| ubiquitin C | AA600188 | 0.734 | 0.00290 | 0.755 | 0.0252 |
| ubiquitin carrier protein | AI571293 | 0.794 | 0.00146 | 0.750 | 0.0158 |
| ubiquitin specific protease 14 (tRNA-guanine | NM_005151 | 0.794 | 0.01580 | 0.562 | 0.0311 |
| transglycosylase) | | | | | |
| ubiquitin specific protease 9, X chromosome | NM_004652 | 0.779 | 0.00582 | | |
| (fat facets-like Drosophila) | | | | | |
| ubiquitin-activating enzyme E1C (UBA3 | AL117566 | 0.742 | 0.01442 | 0.665 | 0.0277 |
| homolog, yeast) | | | | | |
| ubiquitin-conjugating enzyme E2A (RAD6 | NM_003336 | 0.702 | 0.00105 | 0.718 | 0.0370 |
| homolog) | | | | | |
| ubiquitin-conjugating enzyme E2D 1 (UBC4/5 | AI816068 | 0.768 | 0.00096 | 0.629 | 0.0464 |
| homolog, yeast) | | | | | |
| ubiquitin-conjugating enzyme E2G 1 (UBC7 | D78514 | 0.799 | 0.02525 | | |
| homolog, C. elegans) | | | | | |
| ubiquitin-conjugating enzyme E2N (UBC13 | D83004 | 0.715 | 0.00318 | | |
| homolog, yeast) | | | | | |
| ubiquitin-like 1 (sentrin) | U61397 | 0.740 | 0.00300 | | |

TABLE 4

| | | Dentate | | CA3 | |
|---|---|---|---|---|---|
| Gene Description | Genbank | Ratio | P Value | Ratio | P Value |
| ATPase, H+ transporting, lysosomal 13 kD, V1 | AW962223 | 0.609 | 0.00053 | 0.475 | 0.04296 |
| subunit G isoform 2 | | | | | |
| ATPase, H+ transporting, lysosomal 21 kD, V0 | D89052 | 0.759 | 0.02063 | 0.734 | 0.02699 |
| subunit c | | | | | |
| ATPase, H+ transporting, lysosomal 31 kD, V1 | X76228 | 0.673 | 0.00003 | 0.634 | 0.04942 |
| subunit E isoform 1 | | | | | |
| ATPase, H+ transporting, lysosomal 34 kD, V1 | H82183 | 0.563 | 0.00031 | 0.531 | 0.04111 |
| subunit D | | | | | |
| ATPase, H+ transporting, lysosomal 38 kDa, | X71490 | | | 0.673 | 0.01133 |
| V0 subunit d isoform 1 | | | | | |
| ATPase, H+ transporting, lysosomal 42 kD, V1 | AI338777 | 0.747 | 0.01640 | | |
| subunit C, isoform 1 | | | | | |
| ATPase, H+ transporting, lysosomal 50/57 kD | AF132945 | 0.718 | 0.00270 | | |
| V1 subunit H | | | | | |
| ATPase, H+ transporting, lysosomal 56/58 kD, | L35249 | 0.748 | 0.00059 | | |
| V1 subunit B, isoform 2 | | | | | |
| ATPase, H+ transporting, lysosomal | NM_001183 | 0.797 | 0.00741 | 0.800 | 0.03972 |
| interacting protein 1 | | | | | |
| Human lysosomal membrane glycoprotein | M58485 | 0.765 | 0.00131 | | |
| CD63 mRNA. | | | | | |
| lipase A, lysosomal acid, cholesterol esterase | X76488 | 0.683 | 0.00227 | 0.719 | 0.03961 |
| (Wolman disease) | | | | | |
| Lysosomal-associated multispanning | U51240 | | | 0.758 | 0.02800 |
| membrane protein-5 | | | | | |
| lysosomal-associated protein transmembrane | D14696 | 0.704 | 0.00196 | | |
| 4 alpha | | | | | |
| sphingomyelin phosphodiesterase 1, acid | X59960 | 0.727 | 0.01428 | | |
| lysosomal (acid sphingomyelinase) | | | | | |

TABLE 5

| | | Dentate | | CA3 | |
|---|---|---|---|---|---|
| Gene Description | Genbank | Ratio | P Value | Ratio | P Value |
| alternative; Homo sapiens rac1 gene. | AJ132695 | 1.295 | 0.0030 | | |
| arachidonate 15-lipoxygenase | M23892 | 1.250 | 0.0085 | | |
| CC chemokine receptor-3; CCR3; Human | U51241 | | | 1.485 | 0.0330 |
| eosinophil eotaxin receptor (CMKBR3) gene, | | | | | |
| complete cds. | | | | | |
| chemokine (C—C motif) ligand 13 | U46767 | | | 1.324 | 0.0490 |
| chemokine (C—C motif) receptor 2 | U03882 | 1.265 | 0.0128 | | |
| chemokine binding protein 2 | U94888 | 1.272 | 0.0284 | | |
| complement component 1, r subcomponent | X04701 | 1.315 | 0.0062 | | |
| complement component 5 receptor 1 (C5a ligand) | M62505 | 1.285 | 0.0072 | | |
| H. sapiens cDNA for TREB protein. | X55543 | 1.300 | 0.0228 | | |
| Human CRFB4 gene, partial cds. | U08988 | | | 1.424 | 0.0429 |
| Human helix-loop-helix protein (HEB) gene, | U35052 | 1.462 | 0.0223 | | |
| promoter region and exon 1. | | | | | |
| interleukin 13 | NM_002188 | 1.250 | 0.0115 | | |
| interleukin 17 (cytotoxic T-lymphocyte-associated | U32659 | 1.271 | 0.0105 | | |
| serine esterase 8) | | | | | |
| interleukin 3 receptor, alpha (low affinity) | M74782 | 1.355 | 0.0059 | 1.439 | 0.0160 |
| interleukin 6 receptor | NM_000565 | 1.256 | 0.0377 | 1.540 | 0.0398 |
| interleukin 8 receptor, beta | AW028346 | 1.311 | 0.0097 | | |
| interleukin 9 receptor | M84747 | 1.267 | 0.0008 | | |
| leukocyte immunoglobulin-like receptor, subfamily | AF004231 | | | 1.299 | 0.0353 |
| B (with TM and ITIM domains), member 2 | | | | | |
| leukocyte-associated Ig-like receptor 1 | NM_002287 | | | 1.670 | 0.0052 |
| Lps; encodes most common amino acid sequence | AF177765 | 1.267 | 0.0099 | | |
| in humans; membrane spanning component of the | | | | | |
| human LPS receptor; human homolog of the | | | | | |
| mouse Lps gene product; Homo sapiens toll-like | | | | | |
| receptor 4 (TLR4) gene, TLR4A allele, complete | | | | | |
| cds. | | | | | |
| mannose-binding lectin (protein C) 2, soluble | X15422 | | | 1.499 | 0.0432 |
| (opsonic defect) | | | | | |
| nuclear factor NF-IL6 (AA 1-345); Human gene for | X52560 | 1.282 | 0.0011 | | |
| nuclear factor NF-IL6. | | | | | |
| prostaglandin-endoperoxide synthase 2 | L15326 | 1.283 | 0.0036 | | |
| (prostaglandin G/H synthase and cyclooxygenase) | | | | | |
| receptor-interacting serine-threonine kinase 2 | AF027706 | 1.250 | 0.0038 | | |
| transcription factor 7 (T-cell specific, HMG-box) | X59870 | | | 1.438 | 0.0178 |
| vitronectin (serum spreading factor, somatomedin | X03168 | 1.266 | 0.0220 | | |
| B, complement S-protein) | | | | | |

TABLE 6

| | | Dentate | | CA3 | |
|---|---|---|---|---|---|
| Gene Description | Genbank | Ratio | P Value | Ratio | P Value |
| adaptor-related protein complex 2, sigma 1 subunit | X97074 | 0.791 | 0.00088 | | |
| adaptor-related protein complex 2, sigma 1 subunit | AB030654 | 0.782 | 0.00864 | | |
| adenosine A1 receptor | L22214 | 0.770 | 0.03475 | | |
| ADP-ribosylation factor 4-like | L38490 | 0.610 | 0.00019 | 0.672 | 0.0212 |
| adrenergic, alpha-1D-, receptor | S70782 | | | 0.706 | 0.0198 |
| amino-terminal enhancer of split; GRG PROTEIN; ESP1 | AC005944 | | | 0.759 | 0.0402 |
| PROTEIN; AMINO ENHANCER OF SPLIT; AES-1/AES-2; | | | | | |
| gp130 associated protein GAM; Homo sapiens | | | | | |
| chromosome 19, cosmid F23613, complete sequence. | | | | | |
| amphiphysin (Stiff-Man syndrome with breast cancer | X81438 | 0.643 | 0.00039 | 0.640 | 0.0442 |
| 128 kD autoantigen) | | | | | |
| amphiphysin (Stiff-Man syndrome with breast cancer | U07616 | 0.627 | 0.00055 | | |
| 128 kD autoantigen) | | | | | |
| amyloid beta precursor protein (cytoplasmic tail) binding | D86981 | | | 0.697 | 0.0152 |
| protein 2 | | | | | |
| brain-derived neurotrophic factor | X60201 | 0.724 | 0.03351 | | |
| cadherin-like 22 | AF035300 | 0.665 | 0.00037 | | |
| calbindin 1, 28 kDa | NM_004929 | 0.633 | 0.00579 | | |
| calnexin | L10284 | 0.765 | 0.00335 | 0.744 | 0.0458 |
| Chrot-Leyden crystal protein | L01664 | 0.742 | 0.00000 | | |
| chromosome 11 open reading frame 8 | NM_001584 | 0.731 | 0.04126 | | |
| coated vesicle membrane protein | AK024976 | 0.729 | 0.00080 | | |
| coatomer protein complex, subunit beta 2 (beta prime) | X70476 | 0.741 | 0.02229 | 0.658 | 0.0395 |
| copine VI (neuronal) | AB009288 | 0.785 | 0.02239 | 0.770 | 0.0255 |
| development and differentiation enhancing factor 2 | AB007860 | 0.758 | 0.00386 | | |
| doublecortin and CaM kinase-like 1 | AB002367 | 0.698 | 0.02760 | | |
| drebrin 1 | D17530 | 0.763 | 0.00047 | | |
| dynein, axonemal, heavy polypeptide 9 | AJ404468 | 0.697 | 0.00726 | 0.698 | 0.0271 |
| dystrophin related protein 2 | U43519 | | | 0.706 | 0.0075 |
| early growth response 3 | X63741 | 0.594 | 0.00346 | 0.560 | 0.0345 |
| ephrin-B3 | U66406 | | | 0.659 | 0.0325 |
| fibroblast growth factor 12 | U66197 | 0.792 | 0.03240 | | |
| fibroblast growth factor 13 | NM_004114 | 0.755 | 0.04986 | | |
| fibroblast growth factor 7 (keratinocyte growth factor) | AI075338 | 0.706 | 0.02709 | | |
| GDP dissociation inhibitor 2 | Y13286 | | | 0.768 | 0.0228 |
| glutamate receptor, metabotropic 3 | X77748 | | | 0.780 | 0.0338 |
| growth arrest and DNA-damage-inducible, alpha | M60974 | 0.674 | 0.02750 | | |
| growth arrest-specific 2 | U95032 | 0.739 | 0.04722 | | |
| growth associated protein 43 | M25667 | 0.609 | 0.00029 | | |
| growth associated protein 43 | F02494 | 0.732 | 0.00127 | 0.686 | 0.0280 |
| growth factor receptor-bound protein 10 | D86962 | 0.782 | 0.03071 | | |
| Homo sapiens cDNA FLJ10863 fis, clone | AK001725 | 0.767 | 0.00400 | | |
| NT2RP4001575, highly similar to Rattus norvegicus | | | | | |
| mRNA for ARE1 protein. | | | | | |
| Homo sapiens vesicle trafficking protein sec22b mRNA, | AF047442 | 0.747 | 0.00253 | | |
| complete cds. | | | | | |
| human alpha-tubulin mRNA, 3′ end. | K00557 | | | 0.699 | 0.0312 |
| Human fibroblast growth factor homologous factor 4 | U66200 | | | 0.796 | 0.0404 |
| (FHF-4) mRNA, complete cds. | | | | | |
| huntingtin-associated protein interacting protein (duo) | NM_003947 | | | 0.731 | 0.0422 |
| inhibitor of DNA binding 2, dominant negative helix-loop- | M97796 | 0.798 | 0.00443 | | |
| helix protein | | | | | |
| insulin-like growth factor 1 receptor | X04434 | 0.729 | 0.00039 | | |
| kinesin family member 3C | AF035621 | 0.798 | 0.02051 | | |
| low density lipoprotein receptor (familial | NM_000527 | 0.631 | 0.00136 | 0.783 | 0.0408 |
| hypercholesterolemia) | | | | | |
| low density lipoprotein-related protein-associated protein | NM_002337 | 0.764 | 0.03547 | 0.639 | 0.0059 |
| 1 (alpha-2-macroglobulin receptor-associated protein 1) | | | | | |
| mannose-6-phosphate receptor (cation dependent) | M16985 | | | 0.696 | 0.0146 |
| mesoderm development candidate 2 | D42039 | | | 0.794 | 0.0371 |
| myelin basic protein | M13577 | 0.663 | 0.00000 | | |
| myelin protein zero (Charcot-Marie-Tooth neuropathy 1B) | D10537 | 0.670 | 0.01637 | | |
| N-ethylmaleimide-sensitive factor attachment protein, | AK023725 | 0.694 | 0.02659 | | |
| gamma | | | | | |
| neural precursor cell expressed, developmentally down- | AW960243 | 0.760 | 0.00108 | 0.662 | 0.0195 |
| regulated 8 | | | | | |
| neuropeptide FF-amide peptide precursor | AF005271 | | | 0.694 | 0.0479 |
| neuropilin 2 | AF016098 | | | 0.728 | 0.0146 |
| phosphotidylinositol transfer protein | D30036 | | | 0.767 | 0.0352 |
| piccolo (presynaptic cytomatrix protein) | AB011131 | 0.756 | 0.00877 | | |
| potassium voltage-gated channel, KQT-like subfamily, | AF074247 | | | 0.662 | 0.0316 |
| member 2 | | | | | |
| predicted protein of HQ2706; Homo sapiens PRO2706 | AF119891 | 0.799 | 0.02872 | | |
| mRNA, complete cds. | | | | | |
| protease, serine, 11 (IGF binding) | Y07921 | 0.720 | 0.00238 | | |
| protein tyrosine phosphatase, receptor-type, Z | M93426 | | | 0.666 | 0.0396 |
| polypeptide 1 | | | | | |
| protocadherin beta 10 | AF131761 | | | 0.650 | 0.0331 |
| protocadherin beta 2 | AF152495 | 0.795 | 0.00434 | | |
| putative; Human neurotrophin-3 (NT-3) gene, complete | M37763 | 0.658 | 0.01027 | | |
| cds. | | | | | |
| RAB33A, member RAS oncogene family | D14889 | 0.627 | 0.00128 | | |
| RAB4A, member RAS oncogene family | NM_004578 | 0.706 | 0.01831 | | |
| RAB5A, member RAS oncogene family | M28215 | 0.777 | 0.04868 | | |
| Rab9 effector p40 | Z97074 | 0.766 | 0.00313 | | |
| radixin | AL137751 | 0.013 | 0.78491 | | |
| Ras-like without CAAX 2 | U78164 | | | 0.501 | 0.0087 |
| Ras-like without CAAX 2 | Y07565 | 0.755 | 0.01070 | 0.495 | 0.0169 |
| retinoblastoma binding protein 7 | U35143 | 0.704 | 0.00153 | 0.662 | 0.0330 |
| Ric-like, expressed in neurons (Drosophila) | Y07565 | 0.735 | 0.00235 | | |
| Ric-like, expressed in neurons (Drosophila) | U78164 | 0.788 | 0.00528 | | |
| roundabout, axon guidance receptor, homolog 1 | AF040990 | | | 0.638 | 0.0301 |
| (Drosophila) | | | | | |
| scrapie responsive protein 1 | AJ224677 | | | 0.645 | 0.0295 |
| sema domain, seven thrombospondin repeats (type 1 and | U52840 | 0.765 | 0.02291 | | |
| type 1-like), transmembrane domain (TM) and short | | | | | |
| cytoplasmic domain, (semaphorin) 5A | | | | | |
| SH3-domain GRB2-like 2 | AF036268 | 0.648 | 0.00113 | 0.551 | 0.0297 |
| sorcin | M32886 | 0.738 | 0.00057 | 0.727 | 0.0218 |
| sorting nexin 1 | U53225 | 0.717 | 0.00549 | | |
| sorting nexin 3 | NM_003795 | 0.650 | 0.01372 | 0.555 | 0.0385 |
| sphingomyelin phosphodiesterase 1, acid lysosomal (acid | X59960 | | | 0.727 | 0.0143 |
| sphingomyelinase) | | | | | |
| stathmin-like 2 | D45352 | 0.675 | 0.04962 | 0.643 | 0.0381 |
| superoxide dismutase 1, soluble (amyotrophic lateral | X02317 | 0.714 | 0.01620 | 0.621 | 0.0488 |
| sclerosis 1 (adult)) | | | | | |
| synaptic glycoprotein SC2 | AAF32373 | 0.624 | 0.01686 | | |
| synaptojanin 1 | AB020717 | 0.705 | 0.01007 | | |
| synaptosomal-associated protein, 25 kD | D21267 | 0.662 | 0.00160 | | |
| syndecan binding protein (syntenin) | AF006636 | 0.695 | 0.00080 | | |
| syndecan binding protein (syntenin) | AF000652 | 0.649 | 0.00097 | | |
| syntaxin 8 | NM_004853 | 0.699 | 0.00152 | | |
| synuclein, alpha (non A4 component of amyloid | L08850 | 0.659 | 0.00127 | | |
| precursor) | | | | | |
| TGFB inducible early growth response | AF050110 | 0.756 | 0.03234 | | |
| transforming growth factor beta-stimulated protein TSC- | AJ222700 | 0.721 | 0.01087 | | |
| 22 | | | | | |
| tubulin, alpha, ubiquitous | AF141347 | | | 0.729 | 0.0273 |
| tyrosine 3-monooxygenase/tryptophan 5-monooxygenase | U54778 | 0.774 | 0.03442 | | |
| activation protein, epsilon polypeptide | | | | | |
| tyrosine 3-monooxygenase/tryptophan 5-monooxygenase | S80794 | 0.738 | 0.01748 | | |
| activation protein, eta polypeptide | | | | | |
| tyrosine 3-monooxygenase/tryptophan 5-monooxygenase | X56468 | 0.657 | 0.00126 | | |
| activation protein, theta polypeptide | | | | | |
| vesicle-associated membrane protein 3 (cellubrevin) | BE379661 | 0.769 | 0.02787 | | |
| voltage-dependent anion channel 1 | L06132 | 0.724 | 0.01818 | | |
| zinc finger protein 183 (RING finger, C3HC4 type) | X98253 | | | 0.780 | 0.0417 |
| zinc finger protein 45 (a Kruppel-associated box (KRAB) | L75847 | 0.624 | 0.04792 | 0.690 | 0.0215 |
| domain polypeptide) | | | | | |

TABLE 7
Binomial probabilities of some gene groups down
regulated by schizophrenia in dentate.

| | Number of | Number | Probability |
|---|---|---|---|
| Group | expressed genes | of hits | of the group⁽^{*}⁾ |
| Ubiquitin | 73 | 10 | 1.4e−2 |
| Ubiquinon | 33 | 7 | 3.5e−3 |
| ATP synthase | 24 | 10 | 7.0e−7 |
| Proteasome | 55 | 14 | 4.5e−6 |
| tyrosine 3-monooxygenase/ | 7 | 3 | 6.8e−3 |
| tryptophan | | | |
| 5-monooxygenase | | | |
| activation protein | | | |

⁽*⁾Hit probability of 0.0617 is assumed.

### EXAMPLE 2

### Deficient Expression of Proteasome, Ubiquitin, and Mitochondrial Genes in Hippocampal Neurons of Multiple Schizophrenia Patient Groups

This example describes additional experiment, in which laser-capture microdissection (LCM) and cDNA microarrays were used to discover gene expression differences in hippocampal neurons for two cohorts of normal controls and cases with schizophrenia. By “cohort” is meant a groups of individuals who share one or more characteristics in a research study and who are followed over time. The discovery of large clusters of co-directionally changing genes that encode for ubiquitin, the proteasome, and mitochondrial and neuronal functions in schizophrenia indicate that dentate gyrus neurons appear to under-express genes that are essential for normal cellular metabolism, protein processing, and neuronal functions.

Laser-captured hippocampal dentate granule neurons from two separate cohorts of normal controls and schizophrenics (9 and 8, cohort 1, and 14 and 15, cohort 2) were examined and compared with bipolar disease (8/group) and major depressive disorder cases (10/group). Group averages of the expression of human genes from the Agilent human 1 cDNA rnicroarray chip relative to a common pool of control samples were determined. Group expression intensities were independently calculated for representative genes using a polymerase chain reaction assay.

The microarray studies revealed in both schizophrenia cohorts decreases in large, overlapping clusters of genes that encode for protein turnover (i.e., proteasome subunits and ubiquitin), mitochondrial oxidative energy metabolism (i.e. isocitrate, lactate, malate, NADH and succinate dehydrogenases; cytochrome C oxidase and ATP synthase) and genes associated with neurite outgrowth, cytoskeletal proteins, and synapse plasticity. These changes were not obtained in cases with bipolar disorder or non-psychotic major depression, or in dentate neurons of rats treated chronically with clozapine. The changes were not associated with patient demographics including age, sex, brain weight, body weight, post-mortem interval, or drug history.

The decreases of genes involved with mitochondrial metabolism, proteasome function, and synaptic transmission in hippocampal neurons are highly consistent with functional brain imaging and other post-mortem measures in schizophrenia. Decreases in energy metabolism and protein processing of hypofunctioning hippocampal neurons allow our identification of drug discovery targets that can reverse the cognitive and sensory processing deficits of schizophrenia.

### Material and Methods

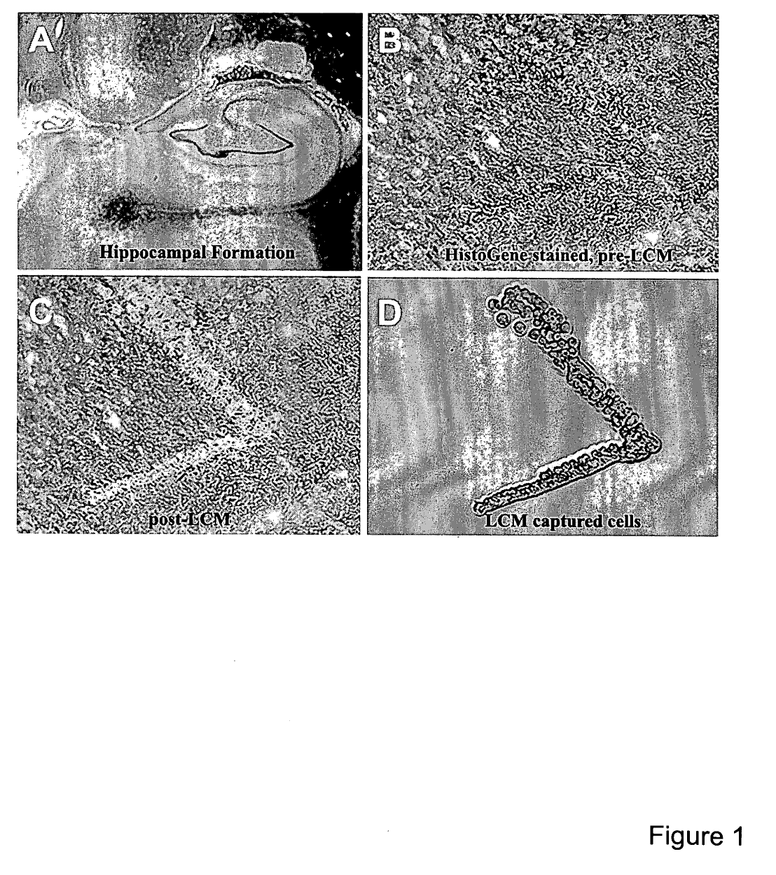

Human tissue: All post-mortem brain tissues used in the present study were obtained from the Stanley Medical Research Institute. The protocols for tissue collection and informed consent were approved by the Institutional Review Board of the Uniformed Services University of the Health Sciences (Torrey et al., 2000). Informed consent from each of the deceased subjects' next of kin was obtained for the use of brain tissue in scientific research. One set of brain sections was obtained from among the Neuropathology Consortium that consists of 60 individuals (n=15 in each of four groups; schizophrenia, bipolar disorder, depression and unaffected controls). A second set of sections was obtained from a cohort of Stanley Foundation non-consortium cases (n=9 schizophrenia and n=9 unaffected controls) and to these were added several samples from cohort 1 (Table 8) whose microarray images failed inclusion criteria in the first study. All cases were diagnosed according to DSM-IV criteria. Details regarding the SMRI brain collection, storage of tissue, and post-mortem diagnosis can be found in Torrey et al (Torrey et al., 2000). The balancing of samples between disease categories according to patient demographics including age, race, body weight, sex, sample pH, and postmortem interval is listed in Table 8.
Preparation of sections: Fourteen μm-thick frozen coronal sections that contained the hippocampus were thaw-mounted onto 1.5×3 inch gelatin-coated microscope slides and stored at −80° C. until use. All subsequent procedures, including neuron capture, RNA processing, and microarray hybridizations were conducted in a blind manner and processed in a counterbalanced order between the two or four diagnosis groups.
Cell capture: Each section was quick-thawed, fixed in 75% ethanol, re-hydrated in dH₂O and stained for two minutes with Arcturus Histogene™ staining solution. The sections were dehydrated in ascending ethanols, placed into xylene for five minutes and air-dried for 15 minutes. Approximately 2000-3000 Nissl-stained dentate granule neurons (FIG. 1) were consistently acquired from 2 or 3 slide-mounted tissue sections of the hippocampus of all cases using a PixCell II-e™ laser-capture microscope (Luo et al., 1999) (Arcturus, Mountain View, Calif.).
RNA amplification. The RNA of sections adjacent to those used for LCM revealed average 28S/18S ratios of 2.06±0.47 (X±SD). These ratios and normal ribosomal band intensities indicated that minimal RNA degradation occurred in these postmortem tissues, and that they were suitable for microarray studies (Bahn et al., 2001). Approximately 1 ng of mRNA extracted from the dentate granule cells of each case underwent two rounds of linear RNA amplification (Van Gelder et al., 1990; Eberwine et al., 1996) using the Arcturus RiboAmp kit (Mountain View, Calif.). This yielded approximately 1 μg and 138 μg of amplified RNA (aRNA) after the first and second round, respectively.
Expression profiling on Agilent cDNA microarrays: 400 ng of aRNA from each sample was reverse-transcribed using the Agilent direct-label cDNA synthesis kit (Palo Alto, Calif.) according to the manufacturer's directions, except that 400 ng of random hexamers was used to prime amplified RNA. Labeled cDNA was purified using QIAquick PCR purification columns (Qiagen, Valencia, Calif.), and concentrated by vacuum centrifugation. The cDNA was suspended in hybridization buffer and hybridized to Agilent Human 1 cDNA microarrays for 17 hours at 65° C. according to the Agilent protocol. Instead of randomly pairing samples from two cases for two channel cDNA arrays (Mirnics et al., 2000), each sample was labeled with cyanine-5 dye and co-hybridized to the same microarray with a common reference sample prepared from a pool of all control samples that was labeled with cyanine-3 dye. Arrays were washed and scanned using an Agilent scanner, using the default settings for cDNA arrays.
Microarray data analysis: RNA failures or poor microarray images occurred in several LCM samples in either experimental cohort. These failures were most commonly due to tissue processing, low amplification yields, and failed chips. The data analysis, therefore, consisted of only the best quality cDNA chips from the Neuropathology Consortium cases (n=9 control, n=8 schizophrenia, n=9 bipolar disorder and n=10 depression) and non-consortium cases (n=15 control and n=14 schizophrenia).

Signal intensities in both channels on all chips were normalized to the global mean of the experiment. Only those genes that produced a mean intensity of at least 300 in at least one of the sample groups were analyzed. For each gene, the log ratio value of each sample/reference was determined and the mean of each patient group was calculated. Logarithms of ratios, referred to as “log ratios”, are commonly used to process two-channel array data because the distribution of ratios is skewed (Quackenbush et al., 2002).
Statistical analysis: To calculate the fold change between the two groups, the mean log ratio of the control group was subtracted from the mean log ratio of the patient group. Raising 2 to the power of this remainder gives the fold change. The formula for computing a ratio of two values from the ratios of each value to a common reference is derived as follows. The common reference comprises a pool of all controlled samples used in the study. If A/R is the ratio of gene expression in schizophrenia to the reference and B/R is the ratio of normal to the reference, then:
   log(A/R)−log(B/R)=log(A)−log(R)−log(B)+log(R)=log(A/B)
   A/B=2^{log}^{₂}(A/B)

The Welch t-test was used to evaluate the statistical significance of disease effects on gene expression. The p values were computed using the t test function implemented in the R statistical software package (See r-project.org on the WorldWideWeb and Venables et al., 2002), with two sets of binary logarithms of sample/reference ratios as the input, e.g., schizophrenia vs. reference and normal vs. reference. Since we were primarily interested in the contrasts between disease cases and normal cases, we have separately compared each disease group to the normal group. t tests are commonly used for such comparisons in the analysis of microarray data (Slonim et al., 2002).

Some of the genes that showed a change in expression levels between diseased and control samples were grouped according to their biological function using the EASE routine and with internally-produced algorithms based on binomial probability computation (Table 9). Such groupings increase confidence in the results when the proportion of genes that change within a group is significantly greater than the proportion of such genes on the entire chip. For example, 10,159 gene probes on the Agilent chip showed a sufficient signal to be considered expressed in the second cohort. About 7.5% of those were altered in schizophrenia, as determined by the criteria of greater than 1.25-fold change and a t test p value less than 0.05. Some of these changes are probably random artifacts due to multiple testing. However, if we identify by name a group of genes that are related to a particular function, such as the proteasome, we see that 32% of them are affected, as determined by the same criteria. The binomial probability computation was used to estimate the probability that such a concentration of “hits” in a particular group of genes could have occurred by chance. Because the size of a functional group of genes is much smaller than the total number of probes on the chip, the binomial probability computation results in p values similar to those obtained with the alternative method, the Fisher exact test used for similar purposes in the EASE (Hosacket al., 2003) and GoMiner software (Zeeberg et al., 2003). The binomial probability computation test is implemented in the R software package (See r-project.org on the Worldwideweb and Venables et al., 2002).
Validation by RT-PCR: Total RNA from ˜2000 re-captured dentate neurons for each sample was subjected to DNase treatment in a 10 μl reaction containing 1 μl 10× DNase I reaction buffer, and 1 Unit DNase I (Invitrogen, Carlsbad, Calif.). The reaction was carried out at room temperature for 10 minutes. One μl of EDTA (25 mM) and 1 μl of random primers (500 μg/ml, Promega, Madison, Wis.) were added to DNase reaction and heated to 70° C. for 15 minutes to simultaneously inactivate the DNase I enzyme and eliminate RNA secondary structure to allow random primer annealing. The sample was placed on ice for two minutes and collected by brief centrifugation. The RNA in the sample was reverse-transcribed into cDNA by the addition of 8 μl of master mix containing 4 μl of 5× first strand buffer, 2 μl DTT (0.1 M), 1 μl dNTP's (10 mM each), and 1 μl SuperScript II (200 U/μl) (Invitrogen, Carlsbad, Calif.), followed by incubation at 42° C. for 45 minutes. The RT reaction was diluted approximately 10-fold with dH₂O and stored at 4° C.

Diluted cDNA (5 μl) added to a 45 μl PCR reaction mixture containing 25 μl of 2× Univeral TaqMan® PCR Master Mix (Applied Biosystems, Foster City, Calif.), 45 picomole of forward and reverse primer, and between 5 and 15 picomole of fluorescently-labeled probe for each specific gene tested. Each sample was subjected to 40 cycles of real time PCR (ABI PRISM® 7900HT, Applied Biosystems, Foster City, Calif.). Fluorescence was measured during each cycle of 2-step PCR alternating between 95° C. for 15 seconds and 60° C. for 1 minute. The threshold cycle (Ct), or cycle number at which signal fluorescence exceeds a preset fluorescence threshold, was compared to a standard curve generated by six, 10-fold serial dilutions of a concentrated reference cDNA standard (prepared from a pool of all control samples). The expression values for each gene were normalized to the average expression levels of three control genes: beta-2-microglobulin (B2M), Dusty protein kinase (DustyPK), and KIAA0582 (an EST). These genes are moderately expressed in dentate granule cells, were unchanged by microarray analysis, and were confirmed to be unchanged by RT-PCR. The normalized relative expression values for all control and treated samples were averaged, and a Student's t test was performed to calculate the statistical significance between these groups.

### Results

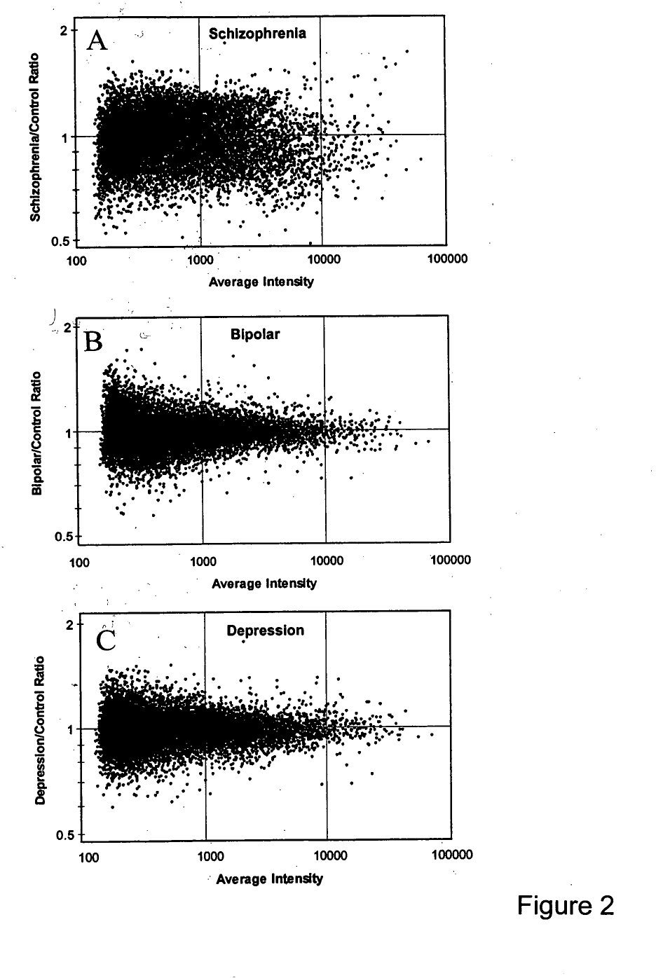

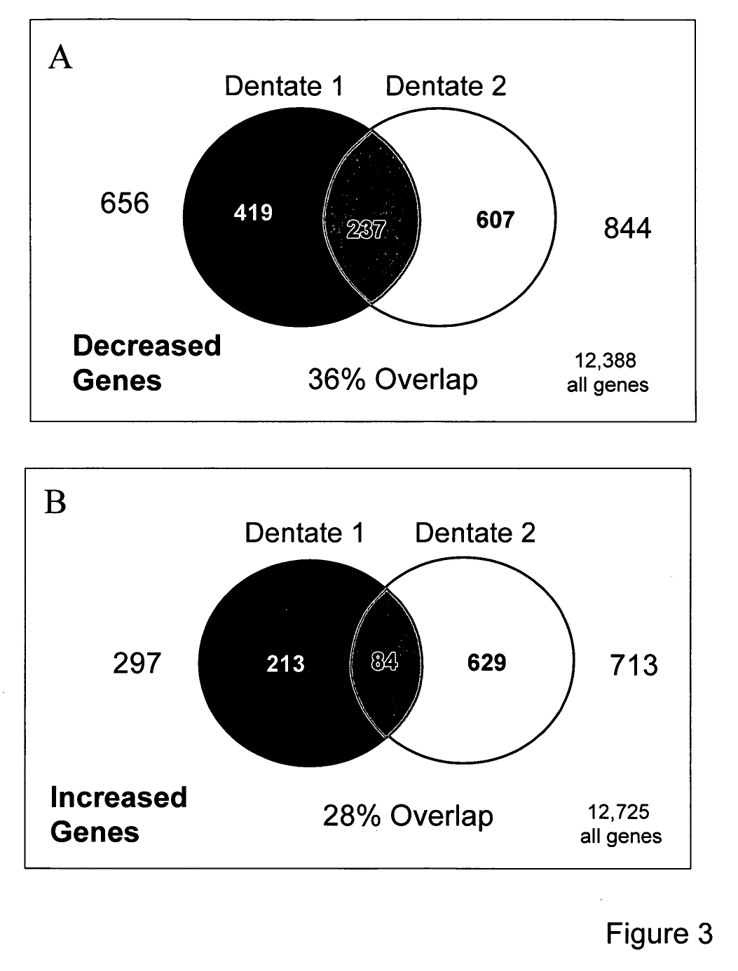

Each microarray was co-hybridized with cyanine-5 labeled cDNA from a case and cyanine-3 labeled cDNA from a pool of all control samples. These two labels allowed the abundance of each gene to be determined for each sample relative to that of the pooled control group (FIG. 2). The average fold change for the bipolar and depression groups relative to the normal group appeared to deviate little from the unity line across a 1000-fold range of gene intensities, and produced gene changes at chance levels regardless of p value. In contrast, the expression of many genes in the schizophrenic cases deviated to a greater extent than in the bipolar or depression cases, and many genes changed significantly from those of the normal controls in the first cohort (n=8-10/group; FIG. 2). A very similar and significant deviation in gene expression from controls occurred in the second cohort of schizophrenic patients (n=14-15/group; data not shown). In each schizophrenia cohort, the incidence of significantly altered genes occurred two- to three-times more frequently than would be expected by chance, at p values of 0.05 and 0.005. Also mitigating against a random nature of these gene changes in schizophrenia was our ability to replicate in the two cohorts many of the decreasing and increasing genes (FIG. 3). Six hundred fifty-six genes were down-regulated in cohort 1, compared to 844 down-regulated genes in cohort 2. Two hundred thirty-seven of these, representing 36% of the down-regulated genes in cohort 1, were also down-regulated in cohort 2. Two hundred ninety seven genes were up-regulated in schizophrenic cases from cohort 1, and 713 genes were up-regulated in cohort 2. Eighty-four of these genes, representing 28% of the up-regulated genes in cohort 1, were replicated in cohort 2. These overlaps were 7- to 5-fold more prevalent than the 5% overlap that would be expected by chance. The fact that over 95% of the genes that changed in both cohorts were co-directional greatly increases the reliability of these findings.

Large sets of genes that decreased in each schizophrenia cohort could be readily grouped into common functional classes. These groupings were independently confirmed by an analysis of changes in gene families using the EASE routine and through programs based upon binomial probabilities (Table 9). The calculation of p values was well below 0.05, such as 10⁻³ to 10⁻⁷, and consistent identification of a common category with the same direction of change in the two cohorts (Table 9), is strong evidence that the effect is distinct and reproducible. For example, among the 57 probes on the Agilent cDNA chip that encode for the proteasome macromolecular complex, 18 were decreased in schizophrenia in the second cohort of cases, (p<0.05,>1.5 fold change). Among the 77 genes on the Agilent chip that encode for members of the ubiquitin pathway, 19 were decreased, while 6 out of the 33 genes that encode for the ubiquinone complexes I-V of the mitochondria were decreased.

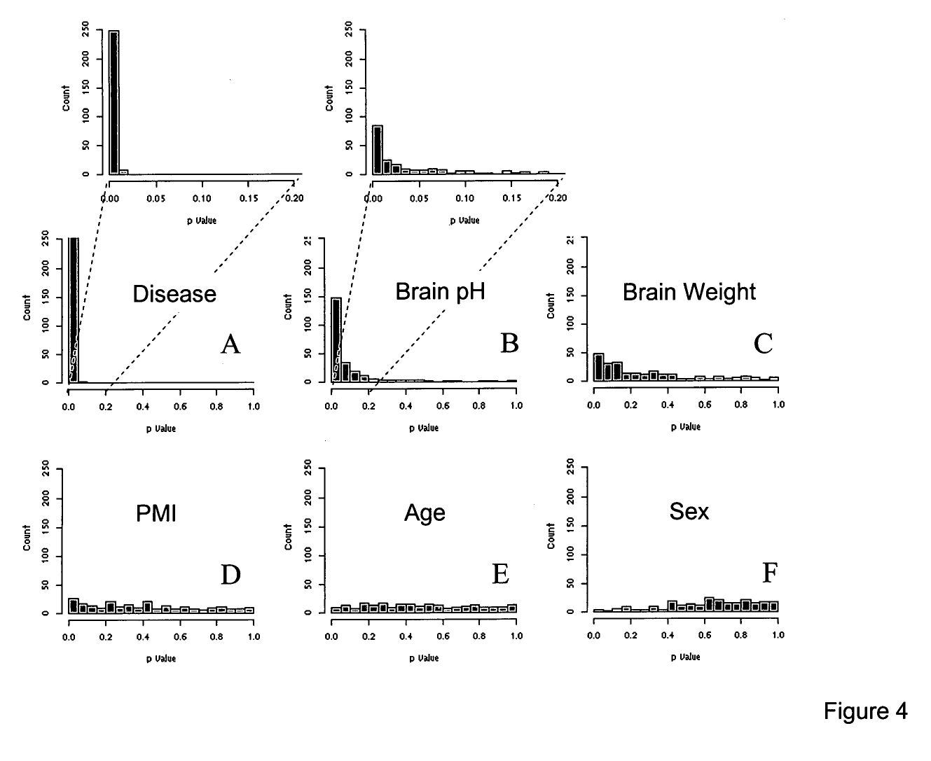

Age, sex, brain pH, brain weight, and body weight (Table 8), and other variables (Torrey et al., 2000) were equally distributed between the normal control and psychiatric cases. Long post-mortem intervals in 3 schizophrenic cases in cohort 1 and in 2 schizophrenic cases in cohort 2 accounted for non-significant but somewhat higher average values compared to controls. Removing these patients had little if any affect the numbers or kinds of genes found to be altered in schizophrenia. Nevertheless, it remained possible that gene expression could vary with one or more of the demographic variables (Vawter et al., 2001; Bahn et al., 2001; Li et al., 2004; Kingsbury et al., 1995; Lehrmann et al., 2003). The variance in gene expression in the normal controls and schizophrenic cases was therefore evaluated by a simple additive model for which the variance of the Log Ratio is a function of the variance contributed by the following factors: Disease, Brain pH, Brain Weight, PMI , Age, Sex, and Body Weight. This model was included in an analysis of variance (ANOVA) of 263 genes that were found by t test to be changed in both cohorts. In this analysis, 44 cases (22 control and 22 schizophrenics) were studied. Numeric factors, such as age (25 to 68 years), PMI (6 to 112 h), brain pH (5.8 to 6.8), brain weight (1260 to 1980 g), body weight (126 to 325 lb), were divided evenly into 5 sub groups. Each sub group was considered as a distinct level for each factor in ANOVA analysis. A histogram of the number of genes whose variance changed as a function of p value of each demographic factor (FIG. 4) revealed how much each factor contributed to the variance in gene expression for the 263 genes. Disease accounted for the majority of variance in gene expression, followed by brain pH and even lesser still, brain weight. Neither post-mortem interval, age, sex (FIG. 4), nor body weight (not shown) contributed to any more than a chance distribution of variability in gene expression.

An ANOVA for the 263 genes was conducted using a model that evaluated the contributions of disease or brain pH to gene variance in both cohorts, according to the model for which the variance in the Log Ratio is a function of the variance contributed by the following factors: Disease and Brain pH. The variance of 70% ofthese genes was not associated with pH in either cohort (data not shown). A significant association with pH was obtained for 20% of the genes in the first cohort, 8% in the second, and only 2% in both. Consistent with the results of the multifactorial ANOVA, the variance of 80% of the genes was associated with disease only in either or both cohorts.

Many of the 263 genes that decreased in both schizophrenia cohorts could be readily grouped into the same functional classes identified by the EASE and binomial routines (Table 9). These included genes encoding for mitochondria and energy metabolism, such as complex I through IV and mitochondrial ATPase (Table 10), and the proteasome and ubiquitin functions (Table 11). Genes important in neuronal plasticity (Table 12) were not identified as a single class by the EASE routine. The TaqMan RT-PCR evaluation of recaptured dentate neurons from 22 control and 22 schizophrenic cases confirmed the changes in 14 of the 20 genes representative of the mitochondria, neuronal plasticity, proteasome, and ubiquitin categories (Table 13). All genes identified as significantly altered in schizophrenia relative to normal controls (n=10/13/group) are listed in Table 14.

Little contribution of antipsychotic treatments to the gene changes in schizophrenia was suggested by use of five sets of observations. Using the Pearson correlation coefficient test, no significant correlation was obtained between the relative change in expression levels of the 263 genes that changed in both cohorts and the cumulative lifetime antipsychotic exposure of the cases.

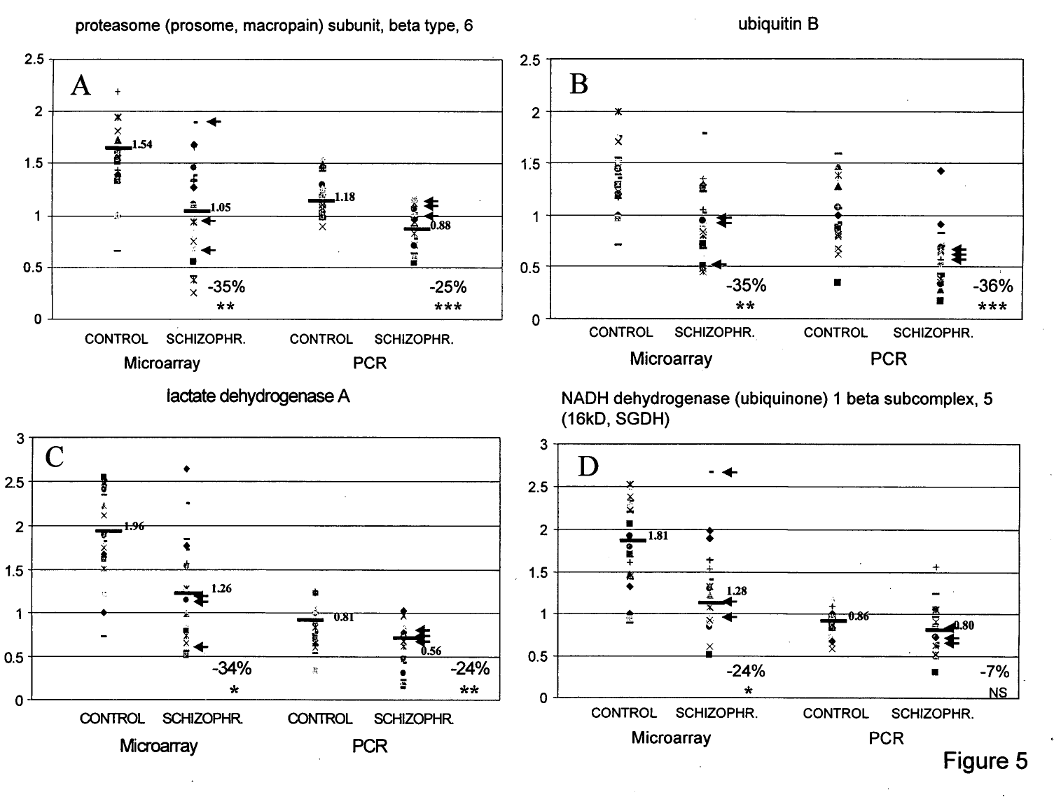

Second, antipsychotics were also taken by the bipolar cases and, though doses were lower than those of the schizophrenics (Table 8), the gene changes in the bipolar cases failed to exceed chance levels or show significant overlap with the schizophrenics. Third, among the cases in both cohorts, three schizophrenics who were medication-free from several weeks to over 30 years prior to death contributed about equally to the gene changes we observed. This is illustrated by arrows for 4 genes representative of the mitochondrial, proteasome, and ubiquitin functions (FIG. 5). Fourth, the pharmacologically complex antipsychotic drug, clozapine, was used by half of the schizophrenic patients, yet a segregation of patients into those who were and were not administered clozapine produced no apparent segregation of changes for these four genes, as evidenced by their random distribution among the expression values for schizophrenic cases.

The potential contributions of antipsychotic treatments to the gene changes in schizophrenic hippocampal dentate granule neurons were further evaluated in male Sprague-Dawley rats (10/group) that received a daily intraperitoneal injection for 21 days of the saline vehicle (10 ml/kg) or clozapine (30 mg/kg). Rats were sacrificed 24 hours after the last injection because this post-injection duration was found to change the expression of more genes compared to a 2 hr survival after clozapine. Hippocampal dentate granule neurons were captured by LCM and processed identically to the human material, using one Agilent 60-mer rodent oligo microarray chip per sample. Compared to vehicle-treated rats, and more than seen with a single clozapine injection, the chronic clozapine-treated rats showed a change in the expression of far more genes than would be expected by chance. However, very few of these genes changed in the same direction as in the dentate granule cells of the schizophrenic cases. Using Locus Link ID numbers for the Agilent human 1 cDNA and Agilent 60-mer rodent oligo microarray chip, 2084 pairs of genes were identified as common to these two chips. Sixty-five of these common genes were among the 263 genes that changed in both cohorts. Among these 65 genes, 15 (23%) changed in the dentate granule neurons from both cohorts and the chronic clozapine-treated rats (p<0.05). Only one of these, proteasome (prosome, macropain) subunit, beta type, 6, is present in Table 10, 11 or 12. The remaining 14 genes were from a longer list of genes that were not among the classes represented in these tables. Interestingly, the proteasome gene was among 4 of the 15 genes that increased in response to clozapine but was decreased in schizophrenia.

Specificity of Gene Changes to Schizophrenia

The gene expression changes described here are disease-specific to schizophrenia for several reasons. First, these changes were not seen in cases with bipolar disease or depression, for which gene expression changes did not exceed chance levels. Also, the gene changes in schizophrenia are not associated with drug abuse or medications. A history of alcohol abuse or dependence was reported for only two of the schizophrenics from the consortium cases but was present in six of the 10 bipolar cases, 4 of the 10 depressed cases, and in several of the control cases. The lack of an alcohol-related effect in generating the changes observed in schizophrenia is important because chronic alcohol abuse or dependence affects mitochondrial, ubiquitin, and proteasome genes in the temporal cortex (Sokolov et al., 2003). While some of these genes overlapped with the consistently down-regulated genes reported here, many of them are increased by alcohol (Sokolov et al., 2003). The lack of concomitant medication effects on the disease signature reported here is also suggested by the fact that three patients who were medication-free from several weeks to over 30 years prior to death contributed about equally to the gene changes observed. Also, the cumulative lifetime antipsychotic exposure of the cases did not correlate with the number of gene changes, and chronic clozapine failed to affect gene homologues in rat dentate granule neurons. Based on these results, and because no other psychiatric drug besides clozapine was given to more than a few of the schizophrenic patients, the possibility of a contribution by concomitant drugs or medications to the gene expression changes observed here can be safely dismissed.

Another demographic variable that has been reported to correlate with brain mRNA expression is brain tissue pH (Li et al., 2004; Kingsbury et al., 1995; Johnston et al., 1997; Harrison et al., 1995). Because of this observation, brain pH was counterbalanced between all groups in the present studies, and brain tissue pH was found to have contributed little to the statistical significance of the gene changes reported here. However, the change in so many genes involved in proton transport suggests that a physiological relationship may exist between brain tissue pH and the degree of gene change. Interestingly, the relatively small number of gene expression changes that correlate with pH were positively correlated, such that their expression decreased with decreases in brain tissue pH. A similar positive correlation has been identified in the brains of non-psychiatric individuals (Li et al., 2004; Kingsbury et al., 1995; Johnston et al., 1997; Harrison et al., 1995), where lower pH was associated with decreases in gene expression and RNA quality. However, the patients that contributed the most to these relationships in those studies (Li et al., 2004; Kingsbury et al., 1995; Johnston et al., 1997; Harrison et al., 1995) experienced prolonged agonal conditions of anoxia, respiratory arrest, or coma. In contrast, samples in the study reported herewere included only if they were obtained from patients who did not experience such prolonged agonal stress, contained RNA of equal, high quality across the groups, and were balanced for brain pH. It is possible that the decreases in brain pH and gene expression are related at a physiological level, since many of the genes that showed this co-variation are involved in mitochondrial proton transport.

Cigarette smoking could also be a factor in the gene changes we saw, as it could alter tissue pH or affect genes linked to nicotinic receptor mechanisms. Smoking data is available for 75% of the cases in cohort 1 and for 65% of the cases in cohort 2. In each diagnostic group of these cohorts, however, the proportion of cases that definitely smoked at the time of death was similar. Thus any effect of smoking would probably have affected gene expression equally across diagnostic groups. Furthermore, there was no significant difference in pH levels between those cases that smoked at the time of death and those that did not smoke, nor did pH vary significantly between diagnostic groups.

The gene signatures of the present invention can be used to determine if an individual is afflicted with schizophrenia. The determination can be made by conducting an analyses of the patient's genes to determine if any of the gene signatures SEQ ID NOS: 1-249 of the present invention are present

### Discussion and Conclusions

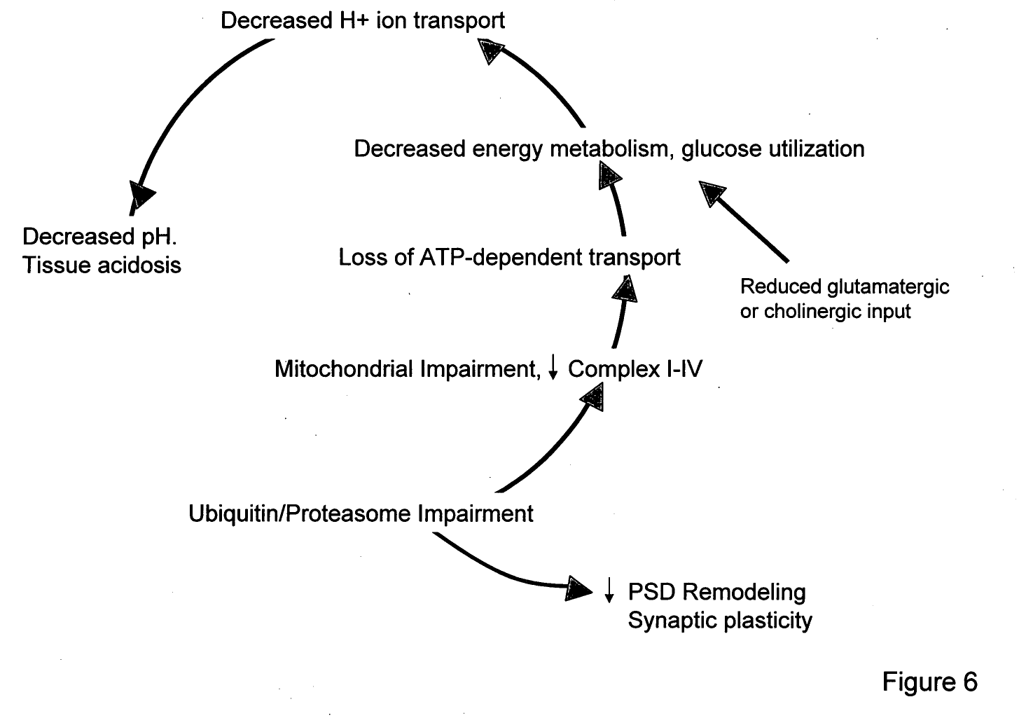

The most consistent and robust gene decreases are those of the various proteasome subunits and for ubiquitin, including ubiquitin-conjugating enzymes. Neuronal ubiquitin (Murphey et al., 2002; Hegde et al., 2002), proteasome (Pak et al., 2003) and the ubiquitin-proteasome system (Ehlers et al., 2003; Speese et al., 2003) control the assembly, connectivity, function, and signaling of the synapse, including regulation of ligand-gated neurotransmitter internalization and turnover of pre- and postsynaptic proteins (Ehlers et al., 2003; Speese et al., 2003). Our observations of decreases in many genes that encode for neuronal plasticity and synaptic functions are consistent with the many reports of synaptic pathology in schizophrenia including microarray-based studies of the frontal cortex (Mimics et al., 2000; Vawter et al., 2001; Bahn et al., 2001; Knable et al., 2001; Hemby et al., 2002). The ability of proteasome inhibitors to deplete neuronal energy reserves and increase neuronal vulnerability to free radical-generators (Hoglinger et al., 2003) suggests that impaired ubiquitin/proteasome functions may weaken the hippocampal synapse by compromising both energy production and synaptic functions (FIG. 6).

The chronic administration of clozapine, haloperidol, or fluphenazine, increases some of the same genes that were decreased in schizophrenia. These include cytochrome C oxidase, which is increased in the hippocampus and frontal cortex of rats treated with these drugs (Prince et al., 1997(a); Prince et al., 1998; Prince et al., 1997(b)), and whose decrease by the psychotomimmetic drugs PCP or methamphetamine is prevented by clozapine and fluphenazine (Prince et al., 1997(b); Prince et al., 1998). Haloperidol enhances and normalizes the utilization of glucose (Holcomb et al., 1996; Desco et al., 2003) and N-acetylasparate (Bertolino et al., 2001) in the schizophrenic brain. Thus, increases in mitochondrial function and glucose utilization may contribute to the therapeutic efficacy of antipsychotic drugs. This hypothesis is supported by the ability of glucose consumption to reverse some of the cognitive deficits in schizophrenia (Stone et al., 2003; Dwyer et al., 2003).

Others have proposed that mitochondrial dysfunction may explain the psychopathology of schizophrenia (Marchbanks et al., 1995; Maurer et al., 2001; Ben-Shachar et al., 2002). This hypothesis is based on decreases in electron transport by cytochrome oxidase and cytochrome C reductase in medicated and unmedicated schizophrenics (Maurer et al., 2001; Whatley et al., 1996; Cavelier et al., 1995). Decreases in these same genes were confirmed in the present report. The many other decreases in genes involved in electron transport and mitochondrial function support a significant role for hippocampal mitochondrial dysfunction in schizophrenia. These decreases are consistent with decreased energy metabolism, glucose utilization (Tamminga et al., 1992; Dickey et al., 2002; Bertolino et al., 1996; Buchsbaum et al., 1990; Nudmamud et al., 2003), and neuronal metabolism in the hippocampus of live schizophrenic patients (Fannon et al., 2003; Bertolino et al., 1996), including decreases in cytochrome c oxidase in post-mortem striatum (Prince et al., 2000). Intellectual and emotional impariments, but not motoric impairments, of schizophrenics correlate strongly and significantly with decreases in striatal cytochrome oxidase (Prince et al., 2000) and with depressed cortical glucose utilization (Buchsbaum et al., 2002). Our findings are also consistent with the 20-30% decreased number of mitochondria in striatal neurons (Kung et al., 1999) and mitochondrial number and volume of striatal and frontal cortex oligodendroglia in schizophrenia (Uranova et al., 2001). These mitochondrial decreases, and the 20-35% decreases in the expression of mitochondrial genes reported here, might be expected to impair overall mitochondrial function. Deficits in mitochondrial metabolism and glucose utilization of dentate gyrus neurons could also result from deficiencies in the depolarizing influences of excitatory glutamatergic (Tsai et al., 1995) or acetylcholinergic inputs (Freedman et al., 2000) proposed for schizophrenia. This scheme is summarized in FIG. 6. A study of whether the gene decreases in schizophrenia can be mimicked in rats by glutamatergic or cholinergic deafferentation could help answer this question and help identify novel pharmacological targets for therapeutic intervention.

Structural and functional deficits of the hippocampus are well-documented in schizophrenia (Benes et al., 2000; Jessen et al., 2003; Tamminga et al., 1992; McCarley et al., 1999; Fannon et al., 2003; Bertolino et al., 1996; Weinberger et al., 1999; Arnold et al., 1996; Velakoulis et al., 2001; Freedman et al., 2000; Buchsbaum et al., 2002; Knable et al., 2004; Lauer et al., 2003). The impairments in cognition, attention, affect, and working memory are relatively persistent features of this disorder and are believed to result in part from hippocampal neuron dysfunction (Weinberger, 1999; Bertolino et al., 2001). Abnormalities of hippocampal dentate granule and CA3 pyramidal neurons (Arnold et al., 1996; Byne etal., 2002; Harrison et al., 1999; Freedman et al., 2000) point to their likely involvement in the cognitive, mnemonic, and affective components of schizophrenia (Benes et al., 2000; Weinberger et al., 1999). Indeed, schizophrenia is perhaps best-characterized by the early-appearing and persisting deficits in cognition, attention, affect, and working memory, recognized by Kraeplin and Bleuler as “dementia praecox” (Kraeplin et al., 1971; Bleuler et al., 1950). It is reasonable to speculate that such deficits in cognitive functions could result from metabolic and protein processing deficits in brain areas like the hippocampus. It remains to be seen whether other homogeneous populations of brain neurons, such as those in the frontal cortex, will demonstrate similar alterations in gene expression as reported described here. A very recent study identified deficits in mitochondrial gene and protein levels in schizophrenia frontal cortex (Bahn et al., 2004). The present results provide a genomic profile of schizophrenia in which hippocampal neurons contain less MRNA for the basic biochemical functions of energy and protein metabolism, and neuronal plasticity. The discovery of compounds that produce a reciprocal change in these same genes may yield novel antipsychotics that address at least some of the core deficits of schizophrenia.

TABLE 8

| | | | | | | Brain | Body | Lifetime |
|---|---|---|---|---|---|---|---|---|
| | | | | | Brain | Weight | Weight | Antipsychotics |
| Description | Case ID # | Age | Sex | PMI | pH | (g) | (g) | (mg) |
| COHORT 1 | | | | | | | | |
| Control | 1 | 53 | M | 28 | 6.2 | 1400 | 133 | 0 |
| Control | 2 | 44 | M | 10 | 6.4 | 1510 | 198 | 0 |
| Control | 3 | 41 | M | 11 | 6 | 1305 | | 0 |
| Control | 4 | 42 | M | 27 | 6.6 | 1500 | 280 | 0 |
| Control | 5 | 57 | F | 26 | 6 | 1400 | 154 | 0 |
| Control | 6 | 52 | M | 28 | 6.5 | 1700 | 191 | 0 |
| Control | 7 | 44 | F | 25 | 6.3 | 1490 | 259 | 0 |
| Control | 8 | 59 | M | 26 | 6.4 | 1560 | 176 | 0 |
| Control | 9 | 52 | M | 8 | 6.5 | 1840 | 188 | 0 |
| Average | | 49.3 | 7M/2F | 21 | 6.3 | 1523 | 197 | 0 |
| Stdev | | 6.7 | | 8.6 | 0.2 | 163 | 50 | 0 |
| Schizophrenic | 10 | 56 | F | 12 | 6.4 | 1420 | 142 | 150000 |
| Schizophrenic | 11 | 30 | F | 60 | 6.2 | 1430 | 141 | 6000 |
| Schizophrenic | 12 | 52 | M | 61 | 6 | 1530 | 174 | 9000 |
| Schizophrenic | 13 | 30 | M | 32 | 5.8 | 1620 | 199 | 50000 |
| Schizophrenic | 14 | 62 | F | 26 | 6.1 | 1270 | 135 | 50000 |
| Schizophrenic | 15 | 60 | M | 31 | 6.2 | 1340 | 158 | 80000 |
| Schizophrenic | 16 | 32 | M | 19 | 6.1 | 1590 | 234 | 15000 |
| Schizophrenic | 17 | 25 | M | 32 | 6.6 | 1555 | 132 | 4000 |
| Average | | 43.4 | 5M/3F | 34.1 | 6.2 | 1469 | 164 | 45500 |
| Stdev | | 15.5 | | 17.7 | 0.2 | 125 | 36 | 50279 |
| Bipolar Disorder | 18 | 25 | F | 24 | 6.4 | 1540 | 157 | 7500 |
| Bipolar Disorder | 19 | 48 | F | 22 | 5.8 | 1260 | 189 | 32000 |
| Bipolar Disorder | 20 | 37 | F | 29 | 6.5 | 1130 | 106 | 1250 |
| Bipolar Disorder | 21 | 57 | M | 19 | 6.2 | 1140 | 193 | 60000 |
| Bipolar Disorder | 22 | 34 | M | 23 | 6.3 | 1523 | 184 | 7000 |
| Bipolar Disorder | 23 | 48 | M | 13 | 6.1 | 1540 | 173 | 200 |
| Bipolar Disorder | 24 | 31 | M | 28 | 6.3 | 1680 | 210 | 30000 |
| Bipolar Disorder | 25 | 50 | M | 19 | 6.2 | 1380 | 208 | 60000 |
| Bipolar Disorder | 26 | 50 | F | 62 | 6.3 | 1320 | 265 | 0 |
| Average | | 42.2 | 5M/3F | 26.6 | 6.2 | 1424 | 187 | 21994 |
| Stdev | | 10.7 | | 14.2 | 0.2 | 169 | 43 | 24691 |
| Depressed | 27 | 44 | F | 32 | 6.2 | 1410 | 123 | 0 |
| Depressed | 28 | 65 | M | 19 | 6.2 | 1360 | 179 | 0 |
| Depressed | 29 | 52 | M | 12 | 6.5 | 1520 | 247 | 0 |
| Depressed | 30 | 42 | F | 25 | 6.3 | 1340 | 136 | 0 |
| Depressed | 31 | 51 | M | 26 | 6.3 | 1550 | 193 | 0 |
| Depressed | 32 | 42 | M | 7 | 6.2 | 1350 | 227 | 0 |
| Depressed | 33 | 56 | M | 23 | 6.5 | 1240 | 198 | 0 |
| Depressed | 34 | 30 | F | 33 | 6 | 1400 | 130 | 0 |
| Depressed | 35 | 43 | M | 43 | 5.9 | 1460 | | 0 |
| Depressed | 36 | 47 | M | 28 | 6.4 | 1740 | 208 | 0 |
| Average | | 47.2 | 7M/3F | 24.8 | 6.3 | 1437 | 182 | 0 |
| Stdev | | 9.5 | | 10.4 | 0.2 | 140 | 44 | 0 |
| COHORT 2 | | | | | | | | |
| Control | 37 | 48 | M | 12 | 6.25 | 1370 | 234 | 0 |
| Control | 38 | 52 | M | 22 | 6.2 | 1330 | 156 | 0 |
| Control | 39 | 35 | F | 23 | 6.6 | 1340 | 126 | 0 |
| Control | 40 | 54 | M | 22 | 6.78 | 1510 | 212 | 0 |
| Control | 41 | 56 | M | 24 | 6.72 | 1500 | 258 | 0 |
| Control | 42 | 35 | F | 40 | 5.8 | 1560 | 146 | 0 |
| Control | 43 | 68 | F | 13 | 6.3 | 1360 | 136 | 0 |
| Control | 44 | 58 | M | 27 | 6 | 1780 | 186 | 0 |
| Control | 45 | 29 | F | 42 | 6.2 | 1440 | | 0 |
| Control | 46 | 56 | M | 36 | 6.62 | 1980 | 199 | 0 |
| Control | 47 | 49 | M | 46 | 6.5 | 1605 | 220 | 0 |
| Control | 48 | 48 | M | 17 | 6.69 | 1600 | 268 | 0 |
| Control | 49 | 48 | M | 12 | 6.51 | 1410 | 296 | 0 |
| Control | 50 | 34 | M | 23 | 6.79 | 1700 | 265 | 0 |
| Control | 51 | 38 | M | 6 | 6.71 | 1460 | 219 | 0 |
| Average | | 47.2 | 11M/4F | 24.3 | 6.4 | 1530 | 209 | 0 |
| Stdev | | 10.9 | | 12 | 0.3 | 182 | 53 | 0 |
| Schizophrenic | 52 | 44 | M | 50 | 6.5 | 1640 | 172 | 100000 |
| Schizophrenic | 53 | 58 | M | 74 | 6.82 | 1835 | 192 | 30000 |
| Schizophrenic | 54 | 44 | M | 29 | 5.9 | 1500 | 260 | 130000 |
| Schizophrenic | 55 | 35 | M | 35 | 6.5 | 1380 | 325 | 50000 |
| Schizophrenic | 56 | 32 | M | 24 | 6.59 | 1440 | 241 | 40000 |
| Schizophrenic | 57 | 40 | M | 70 | 6.62 | 1500 | 188 | 140000 |
| Schizophrenic | 58 | 45 | F | 52 | 6.51 | 1510 | 180 | 20000 |
| Schizophrenic | 59 | 49 | F | 38 | 6.2 | 1260 | 215 | 150000 |
| Schizophrenic | 60 | 57 | M | 20 | 6.66 | 1590 | 222 | 0 |
| Schizophrenic | 61 | 44 | F | 30 | 6.55 | 1480 | 148 | 200000 |
| Schizophrenic | 62 | 35 | M | 9 | 6.44 | 1415 | 128 | 180000 |
| Schizophrenic | 63 | 32 | M | 112 | 6.59 | 1550 | 158 | 1000 |
| Schizophrenic | 64 | 60 | F | 40 | 6.2 | 1395 | 264 | 0 |
| Schizophrenic | 65 | 31 | M | 14 | 5.8 | 1555 | 284 | 4000 |
| Average | | 43.3 | 10M/4F | 42.6 | 6.4 | 1504 | 213 | 74646 |
| Stdev | | 9.9 | | 27.6 | 0.3 | 136 | 57 | 72756 |

TABLE 9

| | Cohort 1 | | | | Cohort 2 | | | |
|---|---|---|---|---|---|---|---|---|
| | Binomial | Binomial | EASE | EASE | Binomial | Binomial | EASE | EASE |
| | Score | Bonf. | Score | Bonf. | Score | Bonf. | Score | Bonf. |
| Proteasome | 4.5E−06 | 4.1E−02 | 1.0E−03 | 1 | 1.1E−07 | 1.20E−03 | 8.1E−04 | 1 |
| (Proteasome Complex) | | | | | | | | |
| Ubiquitin (Ubiquitin- | 1.4E−02 | 1 | 4.6E−02 | 1 | 3.3E−06 | 3.60E−02 | 1.1E−02 | 1 |
| dependent protein | | | | | | | | |
| catabolism) | | | | | | | | |
| Ubiquinone | 3.5E−03 | 1 | | | 3.5E−02 | 1 | | |
| ATP synthase | 7.0E−07 | 6.4E−03 | | | 1 | 1 | | |
| Energy pathways | | | 1.2E−07 | 1.8E−04 | | | 4.3E−05 | 8.4E−02 |
| Mitochondrion | | | 1.3E−07 | 1.9E−04 | | | 1.3E−11 | 2.6E−08 |

TABLE 10

| | Vendor | | | | | |
|---|---|---|---|---|---|---|
| Gene Probe | Probe | Accession | Cohort 1 | | Cohort 2 | |
| ID | ID | Number | Ratio | P value | Ratio | P value |
| 12458 | 3888832 | D90228 | 0.77 | 0.016 | 0.66 | 0.013 |
| 4755 | 1634342 | AW873466 | 0.67 | 0.004 | 0.72 | 0.040 |
| 751 | 1901073 | AF032455 | 0.69 | 0.002 | 0.66 | 0.041 |
| 5267 | 1459967 | X76228 | 0.67 | 0.001 | 0.70 | 0.035 |
| 597 | 4900592 | U83411 | 1.26 | 0.019 | 1.23 | 0.045 |
| 11907 | 1619292 | AK026633 | 0.72 | 0.045 | 0.65 | 0.017 |
| 6731 | 3585709 | M16462 | 0.80 | 0.014 | 0.80 | 0.040 |
| 11212 | 1519369 | AF112219 | 0.62 | 0.023 | 0.74 | 0.037 |
| 4326 | 3126072 | L32977 | 0.66 | 0.008 | 0.64 | 0.026 |
| 5610 | 3986667 | M59783 | 0.69 | 0.006 | 0.62 | 0.021 |
| 5930 | 1573840 | X83464 | 0.77 | 0.002 | 0.76 | 0.039 |
| 1541 | 3490376 | M25161 | 1.23 | 0.028 | 1.20 | 0.038 |
| 9415 | 2054607 | U07681 | 0.72 | 0.008 | 0.76 | 0.036 |
| 2983 | 4372330 | AK026515 | 0.65 | 0.004 | 0.66 | 0.040 |
| 1922 | 1977053 | AW249275 | 0.62 | 0.002 | 0.58 | 0.011 |
| 2583 | 2899863 | AF067166 | 0.74 | 0.005 | 0.71 | 0.043 |
| 10456 | 2467357 | AF047181 | 0.70 | 0.020 | 0.68 | 0.041 |
| 418 | 2935594 | AF020351 | 0.69 | 0.017 | 0.59 | 0.012 |
| 8148 | 1986109 | U30255 | 0.75 | 0.014 | 0.72 | 0.007 |
| 3715 | 4421909 | J04173 | 0.60 | 0.004 | 0.64 | 0.040 |
| 9177 | 1903759 | AW959460 | 0.78 | 0.036 | 0.57 | 0.004 |
| 880 | 1741214 | NM_000925 | 0.76 | 0.042 | 0.64 | 0.048 |
| 14 | 644927 | AI332708 | 0.65 | 0.012 | 0.70 | 0.043 |
| 1568 | 3745348 | J02683 | 0.67 | 0.010 | 0.63 | 0.029 |
| 4204 | 3804843 | L21936 | 0.72 | 0.012 | 0.71 | 0.037 |
| 2191 | 2458933 | AW247564 | 0.69 | 0.024 | 0.58 | 0.019 |

TABLE 11

| Gene | Vendor | | | | | |
|---|---|---|---|---|---|---|
| Probe | Probe | Accession | Cohort 1 | | Cohort 2 | |
| ID | ID | Number | Ratio | P value | Ratio | P value |

Proteasome

| | | | | | | |
|---|---|---|---|---|---|---|
| 10492 | 1488021 | AF006305 | 0.78 | 0.027 | 0.63 | 0.040 |
| 1045 | 2123183 | BE271628 | 0.76 | 0.028 | 0.65 | 0.036 |
| 11991 | 1872245 | D38047 | 0.68 | 0.008 | 0.69 | 0.046 |
| 3002 | 2057812 | AB003177 | 0.74 | 0.003 | 0.72 | 0.011 |
| 9466 | 2211625 | AA310524 | 0.69 | 0.021 | 0.74 | 0.005 |
| 5380 | 2195309 | AI889267 | 0.72 | 0.019 | 0.53 | 0.005 |
| 9595 | 2989852 | BE264172 | 0.67 | 0.006 | 0.56 | 0.024 |
| 1497 | 4534748 | D29012 | 0.68 | 0.009 | 0.61 | 0.023 |
| 1513 | 5161001 | D29012 | 0.73 | 0.002 | 0.58 | 0.009 |

Ubiquitin

| | | | | | | |
|---|---|---|---|---|---|---|
| 4324 | 2054420 | AI660551 | 0.76 | 0.027 | 0.74 | 0.026 |
| 1581 | 3737319 | AF224669 | 0.80 | 0.011 | 0.61 | 0.009 |
| 7772 | 1599036 | AF076269 | 0.65 | 0.020 | 0.69 | 0.031 |
| 7463 | 3137251 | BE250544 | 0.71 | 0.009 | 0.71 | 0.036 |
| 9051 | 2156453 | X04803 | 0.66 | 0.008 | 0.64 | 0.032 |
| 6469 | 2365530 | AI816068 | 0.81 | 0.030 | 0.77 | 0.046 |
| 10565 | 3340760 | NM_014235 | 1.31 | 0.002 | 1.23 | 0.039 |

TABLE 12

| Gene Probe | Vendor | Accession | Cohort 1 | | Cohort 2 | |
|---|---|---|---|---|---|---|
| ID | Probe ID | Number | Ratio | P value | Ratio | P value |
| 11533 | 2819848 | L22214 | 0.78 | 0.040 | 0.83 | 0.017 |
| 5702 | 3837686 | L12168 | 0.80 | 0.014 | 0.77 | 0.042 |
| 10551 | 1505827 | AB003476 | 0.75 | 0.025 | 0.67 | 0.025 |
| 6131 | 702628 | AF022109 | 1.29 | 0.018 | 1.25 | 0.032 |
| 4823 | 3561540 | S77094 | 0.47 | 0.002 | 0.53 | 0.034 |
| 9282 | 4117578 | NM_001584 | 0.74 | 0.048 | 0.66 | 0.019 |
| 4447 | 1650782 | X70476 | 0.75 | 0.029 | 0.71 | 0.045 |
| 7602 | 1854862 | D17530 | 0.77 | 0.001 | 0.82 | 0.017 |
| 4575 | 1873115 | U50733 | 0.81 | 0.013 | 0.74 | 0.029 |
| 8873 | 4063074 | AJ404468 | 0.70 | 0.009 | 0.68 | 0.014 |
| 10457 | 4244154 | AF035300 | 0.67 | 0.001 | 0.69 | 0.010 |
| 3996 | 2267630 | NM_005544 | 1.23 | 0.046 | 1.22 | 0.043 |
| 382 | 3629462 | AW296221 | 1.26 | 0.022 | 1.33 | 0.025 |
| 7648 | 1649906 | L06237 | 1.22 | 0.015 | 1.37 | 0.004 |
| 7500 | 1754454 | AW960243 | 0.79 | 0.028 | 0.73 | 0.022 |
| 3704 | 4692382 | S41458 | 1.22 | 0.020 | 1.29 | 0.004 |
| 8058 | 1231405 | D14889 | 0.63 | 0.002 | 0.52 | 0.004 |
| 8544 | 926444 | U78164 | 0.81 | 0.033 | 0.69 | 0.035 |
| 8216 | 2287230 | Y07565 | 0.76 | 0.015 | 0.60 | 0.008 |
| 10389 | 1902608 | AK001725 | 0.77 | 0.005 | 0.76 | 0.019 |
| 2635 | 5121313 | U52840 | 0.77 | 0.029 | 0.76 | 0.012 |
| 3950 | 1259691 | AB002372 | 1.36 | 0.003 | 1.43 | 0.013 |
| 6381 | 1890049 | NM_004853 | 0.70 | 0.002 | 0.70 | 0.045 |
| 696 | 5297230 | AF060568 | 1.30 | 0.013 | 1.43 | 0.004 |
| 11609 | 311459 | AF060568 | 1.29 | 0.001 | 1.33 | 0.026 |

TABLE 13

| | | Microarray, | | Microarray, | | TaqMan Q-PCR | |
|---|---|---|---|---|---|---|---|
| | | Cohort 1 | | Cohort 2 | | Cohorts 1 and 2 | |
| | | Fold | | Fold | | Fold | |
| Accession | Category | change | p value | change | p value | change | p value |
| NM_004046 | Mitochon. | 0.93 | 0.530 | 0.70 | 0.055 | 0.68 | 0.00005 |
| NM_001696 | Mitochon. | 0.67 | 0.001 | 0.70 | 0.035 | 0.79 | 0.00784 |
| NM_004929.2 | Neuronal | 0.69 | 0.103 | 0.51 | 0.004 | 0.55 | 0.00059 |
| NM_001865.2 | Neuronal | 1.00 | 0.962 | 0.70 | 0.071 | 0.68 | 0.00197 |
| NM_002045 | Neuronal | 1.06 | 0.454 | 0.67 | 0.052 | 0.55 | 0.00010 |
| NM_005544.1 | Mitochon. | 1.23 | 0.046 | 1.22 | 0.043 | 0.73 | 0.00232 |
| NM_005566.1 | Mitochon. | 0.65 | 0.004 | 0.66 | 0.040 | 0.76 | 0.00232 |
| NM_002492.1 | Mitochon. | 0.70 | 0.020 | 0.68 | 0.041 | 0.93 | 0.37033 |
| NM_002495.1 | Mitochon. | 0.69 | 0.017 | 0.59 | 0.012 | 0.76 | 0.00035 |
| NM_016446.2 | Neuronal | 0.67 | 10E−7 | 0.74 | 0.043 | 0.72 | 0.00029 |
| NM_148976.1 | Proteasome | 0.72 | 0.019 | 0.53 | 0.005 | 0.65 | 0.00003 |
| NM_002798.1 | Proteasome | 0.73 | 0.002 | 0.58 | 0.009 | 0.75 | 0.00001 |
| NM_002823.2 | Neuronal | 1.44 | 0.005 | 1.55 | 0.008 | 0.99 | 0.88322 |
| NM_004794 | Neuronal | 0.63 | 0.002 | 0.52 | 0.004 | 0.70 | 0.00012 |
| NM_004168.1 | Mitochon. | 0.72 | 0.012 | 0.71 | 0.037 | 0.84 | 0.04256 |
| NM_004853 | Neuronal | 0.70 | 0.002 | 0.70 | 0.045 | 0.92 | 0.13930 |
| NM_018955 | Ubiquitin | 0.66 | 0.008 | 0.64 | 0.032 | 0.64 | 0.00017 |
| NM_003338.3 | Ubiquitin | 0.81 | 0.030 | 0.77 | 0.046 | 0.90 | 0.34074 |

TABLE 14

| | Reference | | | Cohort 1 | | Cohort 2 | |
|---|---|---|---|---|---|---|---|
| SEQ ID NO: | Number | GeneProbeID | Accession | Ratio | P Value | Ratio | P Value |
| SEQ ID NO: 1 | NM_006670 | 12441 | Z29083 | 1.235 | 0.0310 | 1.285 | 0.0371 |
| SEQ ID NO: 2 | NM_003815 | 11233 | U41767 | 1.224 | 0.0076 | 1.278 | 0.0162 |
| SEQ ID NO: 3 | NM_005100 | 8284 | AB003476 | 0.748 | 0.0246 | 0.673 | 0.0253 |
| SEQ ID NO: 4 | NM_000019. | 8340 | D90228 | 0.772 | 0.0156 | 0.661 | 0.0134 |
| SEQ ID NO: 5 | NM_004300 | 5216 | NM_004300 | 0.810 | 0.0415 | 0.734 | 0.0337 |
| SEQ ID NO: 6 | NM_000674 | 11533 | L22214 | 0.776 | 0.0401 | 0.827 | 0.0166 |
| SEQ ID NO: 7 | NM_016282 | 3371 | AK001553 | 0.714 | 0.0463 | 0.664 | 0.0116 |
| SEQ ID NO: 8 | NM_006367 | 7888 | L12168 | 0.799 | 0.0140 | 0.773 | 0.0418 |
| SEQ ID NO: 9 | NM_005484 | 3454 | AK001980 | 0.758 | 0.0296 | 0.698 | 0.0026 |
| SEQ ID NO: 10 | NM_006066 | 8600 | AW873466 | 0.673 | 0.0040 | 0.723 | 0.0399 |
| SEQ ID NO: 11 | NM_020299. | 11182 | AF032455 | 0.686 | 0.0023 | 0.663 | 0.0408 |
| SEQ ID NO: 12 | NM_014885 | 3712 | AF132794 | 1.338 | 0.0238 | 1.299 | 0.0394 |
| SEQ ID NO: 13 | NM_003801 | 3883 | NM_003801 | 0.741 | 0.0061 | 0.719 | 0.0058 |
| SEQ ID NO: 14 | NM_001139. | 7002 | AF038461 | 1.210 | 0.0340 | 1.208 | 0.0412 |
| SEQ ID NO: 15 | S80343 | 10686 | S80343 | 0.757 | 0.0478 | 0.692 | 0.0327 |
| SEQ ID NO: 16 | NM_001696 | 2365 | X76228 | 0.671 | 0.0010 | 0.703 | 0.0353 |
| SEQ ID NO: 17 | NM_004323 | 3950 | Z35491 | 0.771 | 0.0116 | 0.768 | 0.0486 |
| SEQ ID NO: 18 | NM_001726 | 10551 | AA884041 | 1.363 | 0.0172 | 1.352 | 0.0403 |
| SEQ ID NO: 19 | NM_007371 | 2264 | D26362 | 1.366 | 0.0007 | 1.381 | 0.0337 |
| SEQ ID NO: 20 | NM_001328 | 12415 | NM_001328 | 0.642 | 0.0287 | 0.601 | 0.0307 |
| SEQ ID NO: 21 | NM_018398 | 2616 | AJ272213 | 0.704 | 0.0213 | 0.628 | 0.0097 |
| SEQ ID NO: 22 | NM_016352 | 11963 | AF095719 | 1.247 | 0.0017 | 1.223 | 0.0116 |
| SEQ ID NO: 23 | NM_003652 | 418 | U83411 | 1.255 | 0.0193 | 1.229 | 0.0451 |
| SEQ ID NO: 24 | NM_001334 | 6131 | N20599 | 0.788 | 0.0495 | 0.751 | 0.0287 |
| SEQ ID NO: 25 | NM_005194. | 2597 | X52560 | 1.239 | 0.0178 | 1.210 | 0.0226 |
| SEQ ID NO: 26 | NM_001254. | 751 | AF022109 | 1.285 | 0.0180 | 1.251 | 0.0318 |
| SEQ ID NO: 27 | NM_145054 | 12017 | AAF50630 | 0.632 | 0.0053 | 0.661 | 0.0060 |
| SEQ ID NO: 28 | NM_006430 | 7197 | U38846 | 0.691 | 0.0246 | 0.574 | 0.0439 |
| SEQ ID NO: 29 | NM_000079 | 12634 | S77094 | 0.470 | 0.0017 | 0.529 | 0.0344 |
| SEQ ID NO: 30 | NM_004872 | 10456 | AA305978 | 0.713 | 0.0386 | 0.688 | 0.0360 |
| SEQ ID NO: 31 | NM_001584 | 9282 | NM_001584 | 0.736 | 0.0483 | 0.662 | 0.0187 |
| SEQ ID NO: 32 | NM_005716 | 1988 | AA581812 | 1.225 | 0.0093 | 1.285 | 0.0263 |
| SEQ ID NO: 33 | NM_004891 | 696 | NM_004891 | 0.770 | 0.0073 | 0.686 | 0.0297 |
| SEQ ID NO: 34 | NM_021188 | 11609 | U90919 | 0.563 | 0.0017 | 0.618 | 0.0377 |
| SEQ ID NO: 35 | NM_004766. | 4447 | X70476 | 0.746 | 0.0285 | 0.709 | 0.0452 |
| SEQ ID NO: 36 | NM_033138 | 10323 | D79413 | 1.283 | 0.0126 | 1.323 | 0.0306 |
| SEQ ID NO: 37 | NM_001847. | 2583 | D21337 | 1.348 | 0.0277 | 1.344 | 0.0216 |
| SEQ ID NO: 38 | NM_001849 | 466 | AL360197 | 0.828 | 0.0386 | 0.720 | 0.0258 |
| SEQ ID NO: 39 | NM_016129 | 8649 | AK001148 | 0.623 | 0.0188 | 0.524 | 0.0028 |
| SEQ ID NO: 40 | NM_005190 | 4947 | M74091 | 0.787 | 0.0401 | 0.725 | 0.0178 |
| SEQ ID NO: 41 | NM_004354 | 6386 | AI271688 | 0.754 | 0.0435 | 0.618 | 0.0040 |
| SEQ ID NO: 42 | NM_000397 | 7493 | X04011 | 0.749 | 0.0016 | 0.701 | 0.0303 |
| SEQ ID NO: 43 | NM_001916 | 11729 | AK026633 | 0.723 | 0.0453 | 0.654 | 0.0174 |
| SEQ ID NO: 44 | NM_019010 | 11212 | X73501 | 1.244 | 0.0116 | 1.204 | 0.0366 |
| SEQ ID NO: 45 | AW351829 | 10444 | AW351829 | 1.221 | 0.0368 | 1.317 | 0.0084 |
| SEQ ID NO: 46 | NM_004088 | 970 | NM_004088 | 1.255 | 0.0264 | 1.281 | 0.0242 |
| SEQ ID NO: 47 | NM_000398 | 2503 | M16462 | 0.801 | 0.0140 | 0.800 | 0.0399 |
| SEQ ID NO: 48 | NM_012135 | 6101 | Y18504 | 0.624 | 0.0083 | 0.586 | 0.0094 |
| SEQ ID NO: 49 | NM_004395 | 7602 | D17530 | 0.769 | 0.0006 | 0.816 | 0.0170 |
| SEQ ID NO: 50 | NM_006400 | 11630 | U50733 | 0.810 | 0.0132 | 0.737 | 0.0290 |
| SEQ ID NO: 51 | NM_004662 | 8873 | AJ404468 | 0.703 | 0.0088 | 0.678 | 0.0140 |
| SEQ ID NO: 52 | L19267. | 1997 | L19267 | 1.247 | 0.0479 | 1.343 | 0.0259 |
| SEQ ID NO: 53 | NM_000109 | 2160 | AA661835 | 0.744 | 0.0215 | 0.656 | 0.0475 |
| SEQ ID NO: 54 | NM_007040. | 12542 | AJ007509 | 1.210 | 0.0002 | 1.217 | 0.0347 |
| SEQ ID NO: 55 | NM_006816. | 11384 | BE254013 | 0.828 | 0.0067 | 0.710 | 0.0170 |
| SEQ ID NO: 56 | NM_001984 | 9805 | AF112219 | 0.624 | 0.0235 | 0.743 | 0.0369 |
| SEQ ID NO: 57 | NM_182647 | 8474 | AA827495 | 0.728 | 0.0070 | 0.752 | 0.0222 |
| SEQ ID NO: 58 | NM_014239 | 2796 | AF035280 | 0.772 | 0.0071 | 0.672 | 0.0084 |
| SEQ ID NO: 59 | NM_003753 | 8558 | AW249334 | 0.815 | 0.0175 | 0.719 | 0.0276 |
| SEQ ID NO: 60 | NM_005236. | 11439 | L77890 | 1.245 | 0.0267 | 1.326 | 0.0357 |
| SEQ ID NO: 61 | NM_002027. | 7902 | L10413 | 0.785 | 0.0345 | 0.804 | 0.0379 |
| SEQ ID NO: 62 | NM_006329 | 1644 | AF112152 | 0.623 | 0.0002 | 0.687 | 0.0142 |
| SEQ ID NO: 63 | NM_004116 | 14 | D38037 | 0.618 | 0.0055 | 0.700 | 0.0048 |
| SEQ ID NO: 64 | NM_004111 | 3887 | BE278623 | 0.758 | 0.0337 | 0.713 | 0.0267 |
| SEQ ID NO: 65 | NM_004816 | 12538 | L27479 | 1.207 | 0.0125 | 1.316 | 0.0043 |
| SEQ ID NO: 66 | NM_004477 | 5447 | AA905219 | 0.789 | 0.0200 | 0.686 | 0.0217 |
| SEQ ID NO: 67 | NM_002092 | 8310 | U07231 | 0.741 | 0.0158 | 0.652 | 0.0340 |
| SEQ ID NO: 68 | NM_002051 | 502 | X58072 | 1.284 | 0.0138 | 1.278 | 0.0237 |
| SEQ ID NO: 69 | NM_014628 | 5102 | NM_014628 | 0.717 | 0.0091 | 0.641 | 0.0233 |
| SEQ ID NO: 70 | NM_004483 | 2372 | D00723 | 0.680 | 0.0139 | 0.691 | 0.0111 |
| SEQ ID NO: 71 | NM_004487. | 8490 | NM_004487 | 0.707 | 0.0236 | 0.657 | 0.0171 |
| SEQ ID NO: 72 | NM_006597 | 11631 | AW249010 | 0.547 | 0.0054 | 0.639 | 0.0336 |
| SEQ ID NO: 73 | NM_004506 | 3143 | NM_004506 | 0.814 | 0.0365 | 0.776 | 0.0306 |
| SEQ ID NO: 74 | NM_177433 | 4554 | U92544 | 0.748 | 0.0144 | 0.753 | 0.0314 |
| SEQ ID NO: 75 | NM_000566 | 11986 | AAD34932 | 1.249 | 0.0288 | 1.286 | 0.0420 |
| SEQ ID NO: 76 | NM_002128 | 10485 | BE266776 | 1.218 | 0.0117 | 1.270 | 0.0225 |
| SEQ ID NO: 77 | NM_005800 | 9330 | X59131 | 0.763 | 0.0278 | 0.543 | 0.0104 |
| SEQ ID NO: 78 | NM_005340 | 2481 | AK026557 | 0.627 | 0.0043 | 0.660 | 0.0410 |
| SEQ ID NO: 79 | NM_007067 | 11199 | AF140360 | 0.767 | 0.0168 | 0.719 | 0.0259 |
| SEQ ID NO: 80 | NM_015980. | 177 | AF113537 | 0.531 | 0.0043 | 0.537 | 0.0410 |
| SEQ ID NO: 81 | NM_024293 | 9735 | AK026155 | 0.751 | 0.0090 | 0.575 | 0.0129 |
| SEQ ID NO: 82 | NM_023924 | 5643 | AK026830 | 0.828 | 0.0196 | 0.784 | 0.0040 |
| SEQ ID NO: 83 | NM_024639 | 12096 | AK027046 | 1.227 | 0.0231 | 1.217 | 0.0422 |
| SEQ ID NO: 84 | AF070536 | 2983 | AF070536 | 1.277 | 0.0212 | 1.359 | 0.0135 |
| SEQ ID NO: 85 | NM_014685 | 4324 | AI660551 | 0.757 | 0.0269 | 0.736 | 0.0258 |
| SEQ ID NO: 86 | NM_006003. | 6058 | L32977 | 0.660 | 0.0076 | 0.636 | 0.0256 |
| SEQ ID NO: 87 | NM_001628. | 11907 | M59783 | 0.687 | 0.0065 | 0.617 | 0.0214 |
| SEQ ID NO: 88 | NM_002038 | 1248 | AK024814 | 0.687 | 0.0491 | 0.776 | 0.0156 |
| SEQ ID NO: 89 | NM_001914 | 3862 | AK026310 | 0.781 | 0.0314 | 0.718 | 0.0432 |
| SEQ ID NO: 90 | NM_003105 | 4354 | U90916 | 1.598 | 0.0442 | 1.904 | 0.0460 |
| SEQ ID NO: 91 | AF282498 | 11926 | AF282498 | 0.647 | 0.0087 | 0.671 | 0.0180 |
| SEQ ID NO: 92 | AF143872 | 10107 | AF143872 | 1.287 | 0.0212 | 1.209 | 0.0142 |
| SEQ ID NO: 93 | NM_017528 | 6579 | AF218007 | 0.821 | 0.0494 | 0.658 | 0.0060 |
| SEQ ID NO: 94 | Z58229 | 2397 | Z58229 | 0.765 | 0.0481 | 0.656 | 0.0138 |
| SEQ ID NO: 95 | NM_004980. | 8341 | AL049557 | 0.785 | 0.0324 | 0.749 | 0.0085 |
| SEQ ID NO: 96 | NM_000175 | 3459 | X83464 | 0.774 | 0.0020 | 0.757 | 0.0386 |
| SEQ ID NO: 97 | AP000500 | 11099 | AP000500 | 1.383 | 0.0222 | 1.339 | 0.0174 |
| SEQ ID NO: 98 | NM_006430. | 6950 | D17080 | 0.714 | 0.0179 | 0.522 | 0.0201 |
| SEQ ID NO: 99 | NM_000786. | 382 | U51684 | 1.314 | 0.0161 | 1.301 | 0.0156 |
| SEQ ID NO: 100 | NM_001780. | 7703 | M58485 | 0.778 | 0.0157 | 0.688 | 0.0291 |
| SEQ ID NO: 101 | NM_003340 | 1581 | AF224669 | 0.805 | 0.0108 | 0.611 | 0.0089 |
| SEQ ID NO: 102 | NM_012212 | 5536 | D49387 | 0.703 | 0.0461 | 0.564 | 0.0040 |
| SEQ ID NO: 103 | NM_003319 | 6868 | X90569 | 1.304 | 0.0110 | 1.542 | 0.0052 |
| SEQ ID NO: 104 | NM_001677. | 4755 | M25161 | 1.232 | 0.0284 | 1.205 | 0.0381 |
| SEQ ID NO: 105 | NM_004820 | 9177 | AF127089 | 1.224 | 0.0082 | 1.271 | 0.0153 |
| SEQ ID NO: 106 | XM_370630 | 1578 | X91192 | 0.794 | 0.0142 | 0.770 | 0.0424 |
| SEQ ID NO: 107 | NM_002823 | 1590 | M67480 | 1.444 | 0.0046 | 1.545 | 0.0082 |
| SEQ ID NO: 108 | NM_005105. | 6697 | AF231512 | 0.745 | 0.0262 | 0.725 | 0.0097 |
| SEQ ID NO: 109 | U72852 | 11096 | U72852 | 1.293 | 0.0230 | 1.254 | 0.0363 |
| SEQ ID NO: 110 | NM_004181 | 7772 | AF076269 | 0.653 | 0.0201 | 0.694 | 0.0311 |
| SEQ ID NO: 111 | NM_018320. | 5992 | AK023139 | 0.796 | 0.0234 | 0.710 | 0.0028 |
| SEQ ID NO: 112 | AK000644 | 1656 | AK000644 | 0.777 | 0.0070 | 0.768 | 0.0165 |
| SEQ ID NO: 113 | NM_022752 | 12046 | AK025712 | 0.707 | 0.0321 | 0.725 | 0.0182 |
| SEQ ID NO: 114 | NM_022720 | 3953 | AK025539 | 1.274 | 0.0094 | 1.218 | 0.0329 |
| SEQ ID NO: 115 | NM_021248 | 10457 | AF035300 | 0.670 | 0.0006 | 0.691 | 0.0097 |
| SEQ ID NO: 116 | NM_005785 | 5222 | NM_005785 | 0.823 | 0.0495 | 0.810 | 0.0349 |
| SEQ ID NO: 117 | NM_001545. | 4332 | X81788 | 0.782 | 0.0119 | 0.730 | 0.0356 |
| SEQ ID NO: 118 | NM_001551 | 4023 | Y08915 | 0.820 | 0.0241 | 0.733 | 0.0415 |
| SEQ ID NO: 119 | M87790 | 8442 | M87790 | 0.761 | 0.0086 | 0.715 | 0.0238 |
| SEQ ID NO: 120 | NM_002221 | 167 | X57206 | 1.226 | 0.0239 | 1.241 | 0.0422 |
| SEQ ID NO: 121 | NM_001567. | 5873 | Y14385 | 0.797 | 0.0496 | 0.800 | 0.0172 |
| SEQ ID NO: 122 | NM_005544 | 12292 | NM_005544 | 1.228 | 0.0459 | 1.215 | 0.0426 |
| SEQ ID NO: 123 | NM_002178. | 6156 | M62402 | 0.616 | 0.0011 | 0.826 | 0.0156 |
| SEQ ID NO: 124 | NM_181469. | 8401 | BE294405 | 0.732 | 0.0067 | 0.687 | 0.0030 |
| SEQ ID NO: 125 | NM_004515 | 674 | AA307289 | 0.771 | 0.0166 | 0.710 | 0.0200 |
| SEQ ID NO: 126 | NM_005530. | 3986 | U07681 | 0.717 | 0.0078 | 0.759 | 0.0357 |
| SEQ ID NO: 127 | NM_075891 | 11356 | AAC70890 | 1.259 | 0.0108 | 1.326 | 0.0127 |
| SEQ ID NO: 128 | NM_014762. | 901 | 013643 | 0.714 | 0.0057 | 0.684 | 0.0199 |
| SEQ ID NO: 129 | NM_015359. | 5285 | D31887 | 0.771 | 0.0088 | 0.695 | 0.0084 |
| SEQ ID NO: 130 | NM_014752 | 10531 | AL047241 | 0.775 | 0.0344 | 0.678 | 0.0216 |
| SEQ ID NO: 131 | XM_291253 | 8481 | D63480 | 0.786 | 0.0289 | 0.654 | 0.0212 |
| SEQ ID NO: 132 | NM_015286. | 7244 | AB002351 | 0.816 | 0.0469 | 0.763 | 0.0218 |
| SEQ ID NO: 133 | NM_014954 | 1539 | AB023202 | 0.762 | 0.0202 | 0.755 | 0.0435 |
| SEQ ID NO: 134 | NM_020868. | 6790 | AB040925 | 0.676 | 0.0120 | 0.756 | 0.0350 |
| SEQ ID NO: 135 | NM_020882 | 11332 | AB040943 | 1.255 | 0.0072 | 1.298 | 0.0008 |
| SEQ ID NO: 136 | NM_003937 | 12458 | AW296221 | 1.264 | 0.0217 | 1.331 | 0.0253 |
| SEQ ID NO: 137 | NM_005566 | 3715 | AK026515 | 0.647 | 0.0042 | 0.659 | 0.0397 |
| SEQ ID NO: 138 | NM_015907 | 9204 | AF061738 | 0.731 | 0.0431 | 0.698 | 0.0165 |
| SEQ ID NO: 139 | NM_002347 | 10406 | NM_002347 | 1.260 | 0.0185 | 1.323 | 0.0105 |
| SEQ ID NO: 140 | NM_016457 | 7648 | M60458 | 0.554 | 0.0310 | 0.760 | 0.0473 |
| SEQ ID NO: 141 | NM_013446 | 2744 | AF117233 | 0.722 | 0.0054 | 0.664 | 0.0244 |
| SEQ ID NO: 142 | NM_005918 | 1922 | AW249275 | 0.623 | 0.0020 | 0.582 | 0.0110 |
| SEQ ID NO: 143 | NM_006699 | 5702 | AF027156 | 0.674 | 0.0076 | 0.767 | 0.0356 |
| SEQ ID NO: 144 | NM_005909. | 7296 | L06237 | 1.215 | 0.0154 | 1.368 | 0.0035 |
| SEQ ID NO: 145 | NM_004526 | 4204 | BE250461 | 0.728 | 0.0044 | 0.765 | 0.0230 |
| SEQ ID NO: 146 | NM_002439 | 9544 | NM_002439 | 1.384 | 0.0002 | 1.228 | 0.0306 |
| SEQ ID NO: 147 | NM_004529. | 4326 | AI110630 | 1.348 | 0.0020 | 1.413 | 0.0487 |
| SEQ ID NO: 148 | NM_021019 | 5171 | M22918 | 0.724 | 0.0016 | 0.627 | 0.0059 |
| SEQ ID NO: 149 | NM_018946 | 2814 | AK001659 | 0.772 | 0.0291 | 0.626 | 0.0164 |
| SEQ ID NO: 150 | NM_177924 | 4286 | AA220921 | 0.700 | 0.0360 | 0.624 | 0.0280 |
| SEQ ID NO: 151 | NM_004546 | 6731 | AF067166 | 0.742 | 0.0049 | 0.714 | 0.0430 |
| SEQ ID NO: 152 | NM_002492. | 9906 | AF047181 | 0.698 | 0.0199 | 0.683 | 0.0414 |
| SEQ ID NO: 153 | NM_002495. | 1541 | AF020351 | 0.689 | 0.0175 | 0.587 | 0.0119 |
| SEQ ID NO: 154 | NM_016446. | 6245 | NM_016446 | 0.674 | 0.0000 | 0.743 | 0.0430 |
| SEQ ID NO: 155 | NM_006156 | 7500 | AW960243 | 0.790 | 0.0284 | 0.733 | 0.0223 |
| SEQ ID NO: 156 | NM_005863. | 558 | AW237438 | 0.776 | 0.0167 | 0.784 | 0.0229 |
| SEQ ID NO: 157 | NM_020202 | 10154 | AF284574 | 0.754 | 0.0076 | 0.741 | 0.0219 |
| SEQ ID NO: 158 | NM_005489. | 5610 | AF124251 | 0.708 | 0.0335 | 0.774 | 0.0251 |
| SEQ ID NO: 159 | NM_002552. | 8337 | NM_002552 | 0.763 | 0.0293 | 0.464 | 0.0216 |
| SEQ ID NO: 160 | NM_002553. | 422 | U92538 | 0.820 | 0.0106 | 0.773 | 0.0151 |
| SEQ ID NO: 161 | NM_006194 | 5939 | U59628 | 1.442 | 0.0295 | 1.365 | 0.0398 |
| SEQ ID NO: 162 | NM_015946 | 5996 | AK025729 | 0.700 | 0.0042 | 0.632 | 0.0043 |
| SEQ ID NO: 163 | NM_000285 | 1194 | J04605 | 0.727 | 0.0367 | 0.598 | 0.0174 |
| SEQ ID NO: 164 | NM_004461 | 2385 | U07424 | 0.751 | 0.0278 | 0.773 | 0.0029 |
| SEQ ID NO: 165 | NM_000283. | 880 | S41458 | 1.219 | 0.0202 | 1.292 | 0.0038 |
| SEQ ID NO: 166 | NM_002631 | 7125 | U30255 | 0.752 | 0.0144 | 0.722 | 0.0066 |
| SEQ ID NO: 167 | NM_002629. | 7898 | J04173 | 0.596 | 0.0036 | 0.640 | 0.0396 |
| SEQ ID NO: 168 | NM_002767 | 9442 | NM_002767 | 0.795 | 0.0326 | 0.609 | 0.0061 |
| SEQ ID NO: 169 | NM_000293. | 1930 | X84908 | 0.738 | 0.0167 | 0.655 | 0.0068 |
| SEQ ID NO: 170 | NM_005837. | 7295 | BE206450 | 0.744 | 0.0107 | 0.636 | 0.0035 |
| SEQ ID NO: 171 | NM_020122. | 11779 | AF155652 | 0.748 | 0.0292 | 0.661 | 0.0199 |
| SEQ ID NO: 172 | NM_002245. | 6586 | U33632 | 0.725 | 0.0479 | 0.684 | 0.0259 |
| SEQ ID NO: 173 | NM_021161 | 11111 | AF279890 | 0.762 | 0.0239 | 0.790 | 0.0225 |
| SEQ ID NO: 174 | NM_000220. | 12116 | U65406 | 1.256 | 0.0314 | 1.297 | 0.0387 |
| SEQ ID NO: 175 | NM_014888. | 2093 | AI491952 | 0.652 | 0.0159 | 0.563 | 0.0204 |
| SEQ ID NO: 176 | NM_002726 | 1263 | X74496 | 0.690 | 0.0282 | 0.700 | 0.0154 |
| SEQ ID NO: 177 | NM_000282 | 277 | S79219 | 0.770 | 0.0295 | 0.699 | 0.0089 |
| SEQ ID NO: 178 | NM_002806 | 10492 | AF006305 | 0.776 | 0.0269 | 0.629 | 0.0404 |
| SEQ ID NO: 179 | NM_002812. | 1045 | BE271628 | 0.755 | 0.0280 | 0.645 | 0.0355 |
| SEQ ID NO: 180 | NM_063010. | 3002 | AB003177 | 0.737 | 0.0026 | 0.716 | 0.0105 |
| SEQ ID NO: 181 | NM_176783 | 9466 | AA310524 | 0.688 | 0.0209 | 0.741 | 0.0048 |
| SEQ ID NO: 182 | NM_002786 | 5380 | AI889267 | 0.721 | 0.0186 | 0.534 | 0.0046 |
| SEQ ID NO: 183 | NM_002798 | 1497 | D29012 | 0.684 | 0.0088 | 0.606 | 0.0230 |
| SEQ ID NO: 184 | NM_014297. | 3996 | NM_014297 | 0.789 | 0.0184 | 0.782 | 0.0119 |
| SEQ ID NO: 185 | NM_006741. | 1273 | U48707 | 0.746 | 0.0417 | 0.711 | 0.0031 |
| SEQ ID NO: 186 | NM_005389. | 1912 | D13892 | 0.633 | 0.0049 | 0.730 | 0.0064 |
| SEQ ID NO: 187 | NM_016145 | 7319 | AW405217 | 0.833 | 0.0053 | 0.826 | 0.0285 |
| SEQ ID NO: 188 | NM_019852 | 6356 | AW959460 | 0.781 | 0.0360 | 0.574 | 0.0039 |
| SEQ ID NO: 189 | NM_005801 | 12691 | AF083441 | 1.238 | 0.0418 | 1.250 | 0.0295 |
| SEQ ID NO: 190 | NM_000925. | 8538 | NM_000925 | 0.757 | 0.0424 | 0.640 | 0.0482 |
| SEQ ID NO: 191 | NM_021252. | 3704 | AK001555 | 1.274 | 0.0307 | 1.446 | 0.0292 |
| SEQ ID NO: 192 | NM_004794 | 8058 | D14889 | 0.632 | 0.0017 | 0.524 | 0.0040 |
| SEQ ID NO: 193 | NM_002873. | 4823 | AF098534 | 0.754 | 0.0209 | 0.667 | 0.0354 |
| SEQ ID NO: 194 | NM_006265. | 3024 | NM_006265 | 0.826 | 0.0354 | 0.774 | 0.0424 |
| SEQ ID NO: 195 | NM_000448. | 9956 | M29474 | 1.241 | 0.0173 | 1.275 | 0.0192 |
| SEQ ID NO: 196 | NM_005056 | 4307 | NM_005056 | 1.355 | 0.0096 | 1.519 | 0.0128 |
| SEQ ID NO: 197 | NM_002939 | 549 | X13973 | 0.818 | 0.0260 | 0.832 | 0.0481 |
| SEQ ID NO: 198 | NM_002930 | 8216 | Y07565 | 0.760 | 0.0147 | 0.605 | 0.0078 |
| SEQ ID NO: 199 | NM_001754 | 9415 | AW963298 | 1.299 | 0.0197 | 1.260 | 0.0074 |
| SEQ ID NO: 200 | NM_080564 | 10389 | AK001725 | 0.773 | 0.0045 | 0.757 | 0.0192 |
| SEQ ID NO: 201 | NM_004589 | 10357 | AI332708 | 0.646 | 0.0118 | 0.696 | 0.0433 |
| SEQ ID NO: 202 | NM_003966 | 2635 | U52840 | 0.771 | 0.0288 | 0.765 | 0.0117 |
| SEQ ID NO: 203 | AX014302 | 8148 | AX014302 | 1.214 | 0.0488 | 1.233 | 0.0287 |
| SEQ ID NO: 204 | AX015317 | 378 | AX015317 | 1.341 | 0.0110 | 1.309 | 0.0398 |
| SEQ ID NO: 205 | AX018011 | 95 | AX018011 | 0.819 | 0.0428 | 0.731 | 0.0294 |
| SEQ ID NO: 206 | AX017279 | 11018 | AX017279 | 1.249 | 0.0327 | 1.326 | 0.0132 |
| SEQ ID NO: 207 | AX014335 | 6676 | AX014335 | 0.812 | 0.0233 | 0.746 | 0.0014 |
| SEQ ID NO: 208 | AX015383 | 4575 | AX015383 | 0.665 | 0.0004 | 0.694 | 0.0117 |
| SEQ ID NO: 209 | AX017516 | 6044 | AX017516 | 0.803 | 0.0461 | 0.681 | 0.0186 |
| SEQ ID NO: 210 | AX014881 | 3744 | AX014881 | 0.710 | 0.0135 | 0.788 | 0.0212 |
| SEQ ID NO: 211 | AX017258 | 2371 | AX017258 | 0.649 | 0.0086 | 0.734 | 0.0360 |
| SEQ ID NO: 212 | AX011691 | 5507 | AX011691 | 0.804 | 0.0046 | 0.787 | 0.0176 |
| SEQ ID NO: 213 | AX015417 | 597 | AX015417 | 0.750 | 0.0069 | 0.638 | 0.0068 |
| SEQ ID NO: 214 | NM_032635 | 11303 | AW473288 | 0.749 | 0.0037 | 0.794 | 0.0463 |
| SEQ ID NO: 215 | NM_014281 | 7929 | AF114818 | 0.799 | 0.0263 | 0.721 | 0.0112 |
| SEQ ID NO: 216 | NM_020320 | 4468 | AK023550 | 0.678 | 0.0060 | 0.699 | 0.0122 |
| SEQ ID NO: 217 | NM_007069 | 10562 | AI375733 | 0.772 | 0.0069 | 0.768 | 0.0476 |
| SEQ ID NO: 218 | NM_005210 | 506 | AAD15549 | 1.318 | 0.0246 | 1.503 | 0.0160 |
| SEQ ID NO: 219 | NM_032964 | 933 | NM_004166 | 0.804 | 0.0113 | 0.736 | 0.0147 |
| SEQ ID NO: 220 | NM_003083 | 10496 | AI453282 | 0.739 | 0.0347 | 0.785 | 0.0353 |
| SEQ ID NO: 221 | NM_006936 | 7501 | AA160893 | 0.741 | 0.0170 | 0.648 | 0.0053 |
| SEQ ID NO: 222 | NM_001152 | 1568 | J02683 | 0.666 | 0.0102 | 0.625 | 0.0291 |
| SEQ ID NO: 223 | NM_004168 | 7837 | L21936 | 0.719 | 0.0119 | 0.707 | 0.0373 |
| SEQ ID NO: 224 | NM_003172 | 5291 | Z35093 | 0.826 | 0.0230 | 0.803 | 0.0255 |
| SEQ ID NO: 225 | NM_014723 | 7360 | AB002372 | 1.360 | 0.0035 | 1.427 | 0.0130 |
| SEQ ID NO: 226 | NM_004853 | 6381 | NM_004853 | 0.704 | 0.0019 | 0.700 | 0.0449 |
| SEQ ID NO: 227 | NM_030752 | 3611 | X52882 | 0.702 | 0.0202 | 0.645 | 0.0444 |
| SEQ ID NO: 228 | NM_006520 | 3139 | NM_006520 | 0.763 | 0.0188 | 0.667 | 0.0293 |
| SEQ ID NO: 229 | NM_007375 | 11757 | AL050265 | 0.680 | 0.0282 | 0.701 | 0.0418 |
| SEQ ID NO: 230 | NM_003187 | 5267 | U21858 | 0.733 | 0.0110 | 0.676 | 0.0162 |
| SEQ ID NO: 231 | NM_006930 | 11621 | BE386888 | 0.655 | 0.0068 | 0.657 | 0.0318 |
| SEQ ID NO: 232 | NM_000545 | 3576 | M57732 | 0.798 | 0.0327 | 0.812 | 0.0179 |
| SEQ ID NO: 233 | NM_006335 | 2191 | AW247564 | 0.695 | 0.0236 | 0.578 | 0.0193 |
| SEQ ID NO: 234 | NM_000365. | 5930 | M10036 | 0.674 | 0.0178 | 0.689 | 0.0274 |
| SEQ ID NO: 235 | NM_012410 | 11722 | AJ245820 | 0.656 | 0.0050 | 0.701 | 0.0083 |
| SEQ ID NO: 236 | NM_003332 | 1619 | AF019562 | 1.262 | 0.0410 | 1.341 | 0.0165 |
| SEQ ID NO: 237 | NM_003985. | 675 | AF097738 | 1.277 | 0.0347 | 1.359 | 0.0211 |
| SEQ ID NO: 238 | NM_018955 | 7463 | BE250544 | 0.715 | 0.0090 | 0.709 | 0.0361 |
| SEQ ID NO: 239 | NM_003338 | 6469 | AI816068 | 0.813 | 0.0300 | 0.766 | 0.0463 |
| SEQ ID NO: 240 | NM_014235 | 10565 | NM_014235 | 1.310 | 0.0020 | 1.232 | 0.0387 |
| SEQ ID NO: 241 | NM_005360 | 11168 | AF055376 | 0.696 | 0.0240 | 0.670 | 0.0132 |
| SEQ ID NO: 242 | NM_003374 | 4958 | L06132 | 0.729 | 0.0218 | 0.627 | 0.0323 |
| SEQ ID NO: 243 | NM_003375 | 9571 | AI015604 | 0.714 | 0.0311 | 0.612 | 0.0382 |
| SEQ ID NO: 244 | NM_001079 | 4890 | L05148 | 1.497 | 0.0023 | 1.428 | 0.0239 |
| SEQ ID NO: 245 | NM_003430 | 11943 | AAA59469 | 1.261 | 0.0458 | 1.296 | 0.0410 |
| SEQ ID NO: 246 | NM_006006 | 3717 | AF060568 | 1.300 | 0.0133 | 1.434 | 0.0039 |
| SEQ ID NO: 247 | NM_003457 | 4911 | AF046001 | 0.778 | 0.0243 | 0.675 | 0.0151 |
| SEQ ID NO: 248 | NM_005674 | 6574 | X82125 | 0.768 | 0.0262 | 0.761 | 0.0015 |
| SEQ ID NO: 249 | NM_005857 | 881 | AW192807 | 0.826 | 0.0346 | 0.657 | 0.0153 |

### REFERENCES CITED

Numerous references are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in the entirety and to the same extent as if each reference was individually incorporated by reference.
Tamminga C A, Thaker G K, Buchanan R, Kirkpatrick B, Alphs L D, Chase T N, Carpenter W T. Limbic system abnormalities identified in schizophrenia using positron emission tomography with fluorodeoxyglucose and neocortical alterations with deficit syndrome. Arch Gen Psychiatry. 1992 July; 49(7):522-30.
Benes F M, Berretta S. GABAergic intemeurons: implications for understanding schizophrenia and bipolar disorder. Neuropsychopharmacology. 2001 July; 25(1):1-27.
Hemby S E, Ginsberg S D, Brunk B, Arnold S E, Trojanowski J Q, Eberwine J H. Gene expression profile for schizophrenia: discrete neuron transcription patterns in the entorhinal cortex. Arch Gen Psychiatry. 2002 July; 59(7):631-40.
Arnold S E, Trojanowski J Q. (1996) Recent advances in defining the neuropathology of schizophrenia. Acta Neuropathol (Berl) 92:217-31
Byne W, Buchsbaum M S, Mattiace L A, Hazlett E A, Kemether E, Elhakem S, Purohit D, Haroutunian V, Jones L (2002) Postmortem assessment of thalamic nuclear volumes in subjects with schizophrenia. Am J Psychiatry 159:59-65.
Harrison P J. (1999) The neuropathology of schizophrenia. A critical review of the data and their interpretation. Brain 122 (Pt 4):593-624
Beasley C L, Zhang Z J, Patten I and Reynolds G P (2002) Selective deficits in prefrontal cortical GABAergic neurons in schizophrenia defined by the presence of calcium-binding proteins. Biol. Psychiatry 52:708-15.
Benes F M. Emerging principles of altered neural circuitry in schizophrenia. Brain Res Brain Res Rev. 2000 March;31(2-3):251-69.
Dickey C C, McCarley R W, Shenton M E. (2002) The brain in schizotypal personality disorder: a review of structural MRI and CT findings. Harv Rev Psychiatry 10:1-15.
McCarley R W, Wible C G, Frumin M, Hirayasu Y, Levitt J J, Fischer I A, Shenton M E. (1999) MRI anatomy of schizophrenia. Biol Psychiatry 45:1099-119
Kishimoto H, Yamada K, Iseki E, Kosaka K, Okoshi T. (1998) Brain imaging of affective disorders and schizophrenia. Psychiatry Clin Neurosci 52 Suppl:S212-4
Benes F M. Alterations of neural circuitry within layer II of anterior cingulate cortex in schizophrenia. J Psychiatr Res. 1999 November-December; 33(6):511-2.
Benes F M, Berretta S. Amygdalo-entorhinal inputs to the hippocampal formation in relation to schizophrenia. Ann N Y Acad Sci. 2000 June; 911:293-304.
Luo L, Salunga R C, Guo H, Bittner A, Joy K C, Galindo J E, Xiao H, Rogers K E, Wan J S, Jackson M R, Erlander M G (1999) Gene expression profiles of laser-captured adjacent neuronal subtypes. Nat Med 5:117-22.
Eberwine J (1996) Amplication of mRNA populations using aRNA generated from immobilized oligo(dT)-T7 primsed cDNA. Biotechniques 20:584-91
Van Gelder R N, von Zastrow M E, Yool A, Dement W C, Barchas J D, Eberwine J H.
Amplified RNA synthesized from limited quantities of heterogeneous cDNA. Proc Natl Acad Sci USA. 1990 March; 87(5):1663-7.
Mimics K, Middleton F A, Stanwood G D, Lewis D A, Levitt P. Disease-specific changes in regulator of G-protein signaling 4 (RGS4) expression in schizophrenia. Mol Psychiatry. 2001 May; 6(3):293-301.
Middleton F A, Mirnics K, Pierri J N, Lewis D A and Levitt P (2002) Gene expression profiling reveals alterations of specific metabolic pathways in schizophrenia J. Neuroscience 22:2718-29.
Hakak Y, Walker J R, Li C, Wong W H, Davis K L, Buxbaum J D, Haroutunian V, Fienberg A A. Genome-wide expression analysis reveals dysregulation of myelination-related genes in chronic schizophrenia. Proc Natl Acad Sci USA. 2001 April 10; 98(8):4746-51.
Mimics K, Middleton F A, Marquez A, Lewis D A, Levitt P. Molecular characterization of schizophrenia viewed by microarray analysis of gene expression in prefrontal cortex. Neuron. 2000 October; 28(1):53-67
Mimmack, J. L. et al. Gene expression analysis in schizophrenia: reprotricuble up-regulation of several members of the apolipiprotein L family located in a high-susceptibility locus for schizophrenia on chromosome 22. Proc. Natl. Acad. Sci. 2002 USA 99: 4680-4685.
Sun Y, Zhang L, Johnston N L, Torrey E F, Yolken R H. Serial analysis of gene expression in the frontal cortex of patients with bipolar disorder. Br J Psychiatry Suppl. 2001 June; 41:s137-41.
Ohnuma T, Tessler S, Arai H, Faull R L, McKenna P J, Emson P C. Gene expression of metabotropic glutamate receptor 5 and excitatory amino acid transporter 2 in the schizophrenic hippocampus. Brain Res Mol Brain Res. 2000 December 28; 85(1-2):24-31.
Sokolov B P, Tcherepanov A A, Haroutunian V, Davis K L. Levels of mRNAs encoding synaptic vesicle and synaptic plasma membrane proteins in the temporal cortex of elderly schizophrenic patients. Biol Psychiatry. 2000 August 1; 48(3):184-96.
Leonard S, Breese C, Adams C, Benhammou K, Gault J, Stevens K, Lee M, Adler L, Olincy A, Ross R, Freedman R. Smoking and schizophrenia: abnormal nicotinic receptor expression. Eur J Pharmacol. 2000 March 30; 393(1-3):237-42.
Richardson-Burns S M, Haroutunian V, Davis K L, Watson S J, Meador-Woodruff J H. Metabotropic glutamate receptor mRNA expression in the schizophrenic thalamus. Biol Psychiatry. 2000 January 1; 47(1):22-8.
Karson C N, Mrak R E, Schluterman K O, Stumer W Q, Sheng J G, Griffin W S. Alterations in synaptic proteins and their encoding mRNAs in prefrontal cortex in schizophrenia: a possible neurochemical basis for ‘hypofrontality’. Mol Psychiatry. 1999 January; 4(1):39-45.
Barbeau D, Liang J J, Robitalille Y, Quirion R, Srivastava L K. Decreased expression of the embryonic form of the neural cell adhesion molecule in schizophrenic brains. Proc Natl Acad Sci USA. 1995 March 28; 92(7):2785-9.
Meador-Woodruff J H, Grandy D K, Van Tol H H, Damask S P, Little K Y, Civelli O, Watson S J Jr. Dopamine receptor gene expression in the human medial temporal lobe. Neuropsychopharmacology. 1994 July; 10(4):239-48.
Schmauss C, Haroutunian V, Davis K L, Davidson M. Selective loss of dopamine D3-type receptor mRNA expression in parietal and motor cortices of patients with chronic schizophrenia. Proc Natl Acad Sci U S A. 1993 October 1; 90(19):8942-6.
Perrett C W, Whatley S A, Ferrier I N, Marchbanks R M. Changes in relative levels of specific brain mRNA species associated with schizophrenia and depression. Brain Res Mol Brain Res. 1992 January; 12(1-3):163-71.
Mengod G, Vivanco M M, Christnacher A, Probst A, Palacios J M. Study of pro-opiomelanocortin mRNA expression in human postmortem pituitaries. Brain Res Mol Brain Res. 1991 May; 10(2):129-37.
Perlstein W M, Dixit N K, Carter C S, Noll D C, Cohen J D: Prefrontal cortex dysfunction mediates deficits in working memory and prepotent responding in schizophrenia. Biol Psychiatry 2003; 53(1):25-38
Benes F M, Berreta S: Amygdalo-entorhinal inputs to the hippocampal formation in relation to schizophrenia. 2000
Jessen F, Scheef L, Germeshausen L, Tawo Y, Kockler M, Kuhn K U, Maier W, Schild H H, Heun R: Reduced hippocampal activation during encoding and recognition of words in schizophrenia patients. Am J Psychiatry 2003; 160(7):1305-12
Tamminga C A, Thaker G K, Buchanan R, Kirkpatrick B, Alphs L D, Chase T N, Carpenter W T: Limbic system abnormalities identified in schizophrenia using positron emission tomography with fluorodeoxyglucose and neocortical alterations with deficit syndrome. Arch Gen Psychiatry 1992; 49(7):522-30
Dickey C C, McCarley R W, Shenton M E: The brain in schizotypal personality disorder: a review of structural MRI and CT findings. Harv Rev Psychiatry 2002; 10(1):1-15
McCarley R W, Wible C G, Frumin M, Hirayasu Y, Levitt J J, Fischer I A, Shenton M E: MRI anatomy of schizophrenia. Biol Psychiatry 1999; 45(9):1099-119
Kishimoto H, Yamada K, Iseki E, Kosaka K, Okoshi T: Brain imaging of affective disorders and schizophrenia. Psychiatry Clin Neurosci 1998; 52 Suppl:S212-4
Fannon D, Simmons A, Tennakoon L, O'Ceallaigh S, Sumich A, Doku V, Shew C, Sharma T: Selective deficit of hippocampal N-acetylaspartate in antipsychotic-naive patients with schizophrenia. Biol Psychiatry 2003; 54(6):587-98
Bertolino A, Nawroz S, Mattay V S, Barnett A S, Duyn J H, Moonen C T, Frank J A, Tedeschi G, Weinberger D R: Regionally specific pattern of neurochemical pathology in schizophrenia as assessed by multislice proton magnetic resonance spectroscopic imaging. Am J Psychiatry 1996; 153(12):1554-63
Weinberger D R: Cell biology of the hippocampal formation in schizophrenia. Biol Psychiatry 1999; 45(4):395-402
Arnold S E, Trojanowski J Q: Recent advances in defining the neuropathology of schizophrenia. Acta Neuropathol (Berl) 1996; 92(3):217-31
Byne W, Buchsbaum M S, Mattiace L A, Hazlett E A, Kemether E, Elhakem S L, Purohit D P, Haroutunian V, Jones L: Postmortem assessment of thalamic nuclear volumes in subjects with schizophrenia. Am J Psychiatry 2002; 159(1):59-65
Velakoulis D, Stuart G W, Wood S J, Smith D J, Brewer W J, Desmond P, Singh B, Copolov D, Pantelis C: Selective bilateral hippocampal volume loss in chronic schizophrenia. Biol Psychiatry 2001; 50(7):531-9
Harrison P J: The neuropathology of schizophrenia. A critical review of the data and their interpretation. Brain 1999; 122 (Pt 4):593-624
Beasley C L, Zhang Z J, Patten I, Reynolds G P: Selective deficits in prefrontal cortical
GABAergic neurons in schizophrenia defined by the presence of calcium-binding proteins. Biol Psychiatry 2002; 52(7):708-15
Mirnics K, Middleton F A, Marquez A, Lewis D A, Levitt P: Molecular characterization of schizophrenia viewed by microarray analysis of gene expression in prefrontal cortex. Neuron 2000; 28(1):53-67
Vawter M P, Barrett T, Cheadle C, Sokolov B P, Wood W H, 3rd, Donovan D M, Webster M, Freed W J, Becker K G: Application of cDNA microarrays to examine gene expression differences in schizophrenia. Brain Res Bull 2001; 55(5):641-50
Bahn S, Augood S J, Ryan M, Standaert D G, Starkey M, Emson P C: Gene expression profiling in the post-mortem human brain—no cause for dismay. J Chem Neuroanat 2001; 22(1-2):79-94
Middleton F A, Mirnics K, Pierri J N, Lewis D A, Levitt P: Gene expression profiling reveals alterations of specific metabolic pathways in schizophrenia. J Neurosci 2002; 22(7):2718-29
Hakak Y, Walker J R, Li C, Wong W H, Davis K L, Buxbaum J D, Haroutunian V, Fienberg A A: Genome-wide expression analysis reveals dysregulation of myelination-related genes in chronic schizophrenia. Proc Natl Acad Sci USA 2001; 98(8):4746-51
Davis K L, Stewart D G, Friedman J I, Buchsbaum M, Harvey P D, Hof P R, Buxbaum J, Haroutunian V: White matter changes in schizophrenia: evidence for myelin-related dysfunction. Arch Gen Psychiatry 2003; 60(5):443-56
Freedman R, Adams C E, Leonard S: The alpha7-nicotinic acetylcholine receptor and the pathology of hippocampal interneurons in schizophrenia. J Chem Neuroanat 2000; 20(3-4):299-306
Bertolino A, Callicott J H, Mattay V S, Weidenhammer K M, Rakow R, Egan M F, Weinberger
DR: The effect of treatment with antipsychotic drugs on brain N-acetylaspartate measures in patients with schizophrenia. Biol Psychiatry 2001; 49(1):39-46
Buchsbaum M S, Shihabuddin L, Hazlett E A, Schroder J, Haznedar M M, Powchik P, Spiegel-Cohen J, Wei T, Singer M B, Davis K L: Kraepelinian and non-Kraepelinian schizophrenia subgroup differences in cerebral metabolic rate. Schizophr Res 2002; 55(1-2):25-40
Knable M B, Barci B M, Webster M J, Meador-Woodruff J, Torrey E F: Molecular abnormalities of the hippocampus in severe psychiatric illness: postmortem findings from the Stanley Neuropathology Consortium. Mol Psychiatry 2004
Luo L, Salunga R C, Guo H, Bittner A, Joy K C, Galindo J E, Xiao H, Rogers K E, Wan J S, Jackson M R, Erlander M G: Gene expression profiles of laser-captured adjacent neuronal subtypes. Nat Med 1999; 5(1):117-22
Ma X J, Salunga R, Tuggle J T, Gaudet J, Enright E, McQuary P, Payette T, Pistone M, Stecker K, Zhang B M, Zhou Y X, Varnholt H, Smith B, Gadd M, Chatfield E, Kessler J, Baer T M, Erlander M G, Sgroi D C: Gene expression profiles of human breast cancer progression. Proc Natl Acad Sci USA 2003; 100(10):5974-9
Jurata L, Bukhman Y V, Charles V D, Capriglione F, Bullard J, Lemire A, Laeng P, Mohammed A, Pham Q, Brockman J A, Altar C A: Comparison of mRNA profiling technologies for identification of psychiatric disease and drug signatures. J. Neurosci. Meth. 2004; In Press
Torrey E F, Webster M, Knable M, Johnston N, Yolken R H: The stanley foundation brain collection and neuropathology consortium. Schizophr Res 2000; 44(2):151-5
Van Gelder R N, von Zastrow M E, Yool A, Dement W C, Barchas J D, Eberwine J H: Amplified RNA synthesized from limited quantities of heterogeneous cDNA. Proc Natl Acad Sci USA 1990; 87(5):1663-7
Eberwine J: Amplification of mRNA populations using aRNA generated from immobilized oligo(dT)-T7 primed cDNA. Biotechniques 1996; 20(4):584-91
Quackenbush J: Microarray data normalization and transformation. Nat Genet 2002; 32 Suppl:496-501
Abramowitz M, Stegun I A: Logarithmic Function. New York: Dover, 1972
Venables W N, Ripley B D: Modem applied statistics with S., Springer, 2002
Slonim D K: From patterns to pathways: gene expression data analysis comes of age. Nat Genet 2002; 32 Suppl:502-8
Hosack D A, Dennis G, Jr., Sherman B T, Lane H C, Lempicki R A: Identifying biological themes within lists of genes with EASE. Genome Biol 2003; 4(10):R70
Zeeberg B R, Feng W, Wang G, Wang M D, Fojo A T, Sunshine M, Narasimhan S, Kane D W, Reinhold W C, Lababidi S, Bussey K J, Riss J, Barrett J C, Weinstein J N: GoMiner: a resource for biological interpretation of genomic and proteomic data. Genome Biol 2003; 4(4):R28
Li J Z, Vawter M P, Walsh D M, Tomita H, Evans S J, Choudary P V, Lopez J F, Avelar A, Shokoohi V, Chung T, Mesarwi o, Jones E G, Watson S J, Akil H, Bunney W E, Jr., Myers R M: Systematic changes in gene expression in postmortem human brains associated with tissue pH and terminal medical conditions. Hum Mol Genet 2004; 13(6):609-16
Kingsbury A E, Foster O J, Nisbet A P, Cairns N, Bray L, Eve D J, Lees A J, Marsden C D: Tissue pH as an indicator of mRNA preservation in human post-mortem brain. Brain Res Mol Brain Res 1995; 28(2):311-8
Lehrmann E, Hyde T M, Vawter M P, Becker K G, Kleinman J E, Freed W J: The use of microarrays to characterize neuropsychiatric disorders: postmortem studies of substance abuse and schizophrenia. Curr Mol Med 2003; 3(5):437-46
Sokolov B P, Jiang L, Trivedi N S, Aston C: Transcription profiling reveals mitochondrial, ubiquitin and signaling systems abnormalities in postmortem brains from subjects with a history of alcohol abuse or dependence. J Neurosci Res 2003; 72(6):756-67
Johnston N L, Cervenak J, Shore A D, Torrey E F, Yolken R H, Cerevnak J: Multivariate analysis of RNA levels from postmortem human brains as measured by three different methods of RT-PCR. Stanley Neuropathology Consortium. J Neurosci Methods 1997; 77(1):83-92
Harrison P J, Heath P R, Eastwood S L, Burnet P W, McDonald B, Pearson R C: The relative importance of premortem acidosis and postmortem interval for human brain gene expression studies: selective MRNA vulnerability and comparison with their encoded proteins. Neurosci Lett 1995; 200(3):151-4
Murphey R K, Godenschwege T A: New roles for ubiquitin in the assembly and function of neuronal circuits. Neuron 2002; 36(1):5-8
Hegde A N, DiAntonio A: Ubiquitin and the synapse. Nat Rev Neurosci 2002; 3(11):854-61
Pak D T, Sheng M: Targeted protein degradation and synapse remodeling by an inducible protein kinase. Science 2003; 302(5649):1368-73
Ehlers M D: Activity level controls postsynaptic composition and signaling via the ubiquitin-proteasome system. Nat Neurosci 2003; 6(3):231-42
Speese S D, Trotta N, Rodesch C K, Aravamudan B, Broadie K: The ubiquitin proteasome system acutely regulates presynaptic protein turnover and synaptic efficacy. Curr Biol 2003; 13(11):899-910
Knable M B, Torrey E F, Webster M J, Bartko J J: Multivariate analysis of prefrontal cortical data from the Stanley Foundation Neuropathology Consortium. Brain Res Bull 2001; 55(5):651-9
Hemby S E, Ginsberg S D, Brunk B, Arnold S E, Trojanowski J Q, Eberwine J H: Gene expression profile for schizophrenia: discrete neuron transcription patterns in the entorhinal cortex. Arch Gen Psychiatry 2002; 59(7):631-40
Hoglinger G U, Carrard G, Michel P P, Medja F, Lombes A, Ruberg M, Friguet B, Hirsch E C: Dysfunction of mitochondrial complex I and the proteasome: interactions between two biochemical deficits in a cellular model of Parkinson's disease. J Neurochem 2003; 86(5):1297-307
Prince J A, Yassin M S, Oreland L: Neuroleptic-induced mitochondrial enzyme alterations in the rat brain. J Pharmacol Exp Ther 1997; 280(1):261-7
Prince J A, Yassin M S, Orel and L: A histochemical demonstration of altered cyto chrome oxidase activity in the rat brain by neuroleptics. Eur Neuropsychopharmacol 1998; 8(1):1-6
Prince J A, Yassin M S, Oreland L: Normalization of cytochrome-c oxidase activity in the rat brain by neuroleptics after chronic treatment with PCP or methamphetamine. Neuropharmacology 1997; 36(11-12):1665-78
Prince J A, Oreland L: Mitochondrial Activity in the Mapping of Functional Brain Changes in Schizophrenia. Restor Neurol Neurosci 1998; 12(2,3):185-193
Holcomb H H, Cascella N G, Thaker G K, Medoff D R, Dannals R F, Tamminga C A: Functional sites of neuroleptic drug action in the human brain: PET/FDG studies with and without haloperidol. Am J Psychiatry 1996; 153(1):41-9
Desco M, Gispert J D, Reig S, Sanz J, Pascau J, Sarramea F, Benito C, Santos A, Palomo T, Molina V: Cerebral metabolic patterns in chronic and recent-onset schizophrenia. Psychiatry Res 2003; 122(2):125-35
Stone W S, Seidman L J, Wojcik J D, Green A I: Glucose effects on cognition in schizophrenia. Schizophr Res 2003; 62(1-2):93-103
Dwyer D, Lu X, Freeman A: Neuronal glucose metabolism and schizophrenia: therapeutic prospects? Expert Rev. Neurotherapeutics 2003; 3(1):89-100
Marchbanks R M, Mulcrone J, Whatley S A: Aspects of oxidative metabolism in schizophrenia. Br J Psychiatry 1995; 167(3):293-8
Maurer I, Zierz S, Moller H: Evidence for a mitochondrial oxidative phosphorylation defect in brains from patients with schizophrenia. Schizophr Res 2001; 48(1):125-36
Ben-Shachar D: Mitochondrial dysfunction in schizophrenia: a possible linkage to dopamine. J Neurochem 2002; 83(6):1241-51
Whatley S A, Curti D, Marchbanks R M: Mitochondrial involvement in schizophrenia and other functional psychoses. Neurochem Res 1996; 21(9):995-1004
Cavelier L, Jazin E E, Eriksson I, Prince J, Bave U, Oreland L, Gyllensten U: Decreased cytochrome-c oxidase activity and lack of age-related accumulation of mitochondrial DNA deletions in the brains of schizophrenics. Genomics 1995; 29(1):217-24
Buchsbaum M S, Nuechterlein K H, Haier R J, Wu J, Sicotte N, Hazlett E, Asarnow R, Potkin S, Guich S: Glucose metabolic rate in normals and schizophrenics during the Continuous Performance Test assessed by positron emission tomography. Br J Psychiatry 1990; 156:216-27
Nudmamud S, Reynolds L M, Reynolds G P: N-acetylaspartate and N-Acetylaspartylglutamate deficits in superior temporal cortex in schizophrenia and bipolar disorder: a postmortem study. Biol Psychiatry 2003; 53(12):1138-41
Prince J A, Harro J, Blennow K, Gottfries C G, Oreland L: Putamen mitochondrial energy metabolism is highly correlated to emotional and intellectual impairment in schizophrenics. Neuropsychopharmacology 2000; 22(3):284-92
Kung L, Roberts R C: Mitochondrial pathology in human schizophrenic striatum: a postmortem ultrastructural study. Synapse 1999; 31(1):67-75
Uranova N, Orlovskaya D, Vikhreva O, Zimina I, Kolomeets N, Vostrikov V, Rachmanova V: Electron microscopy of oligodendroglia in severe mental illness. Brain Res Bull 2001; 55(5):597-610
Tsai G, Passani L A, Slusher B S, Carter R, Baer L, Kleinman J E, Coyle J T: Abnormal excitatory neurotransmitter metabolism in schizophrenic brains. Arch Gen Psychiatry 1995; 52(10):829-36
Lauer M, Beckmann H, Senitz D: Increased frequency of dentate granule cells with basal dendrites in the hippocampal formation of schizophrenics. Psychiatry Res 2003; 122(2):89-97
Kraeplin E, Barclay R M: Dementia Praecox and Paraphrenia. New York, R. E. Krieger, 1971
Bleuler E: Dementia Praecox or the Group of Schizophrenias. New York, International Universities Press, 1950

What is claimed is:

1. A method for diagnosing schizophrenia in an individual, which method comprises:
(a) obtaining a cell or tissue sample from a first individual suspected of having schizophrenia;
(b) determining levels of expression of at least one nucleic acid in the cell or tissue sample, said nucleic acid being a nucleic acid of SEQ ID NOS: 1-249; and
(c) comparing said levels of expression to levels of expression of the nucleic acid from a sample of a second individual who does not have schizophrenia;
wherein a difference in the levels of expression of the nucleic acid in the cell or tissue sample obtained from the first individual, relative to the levels of expression in the second individual, indicates that the first individual has schizophrenia.

2. A method of claim 1, wherein the levels of expression of a plurality of nucleic acids in the cell or tissue sample are determined, said plurality of nucleic acids selected from the group consisting of SEQ ID NOS: 1-249.

3. A method for identifying a compound to treat schizophrenia, which method comprises:
(a) contacting a cell or tissue sample with a test compound;
(b) determining expression, in the cell or tissue sample, of at least one nucleic acid selected from the nucleic acids of SEQ ID NOS: 1-249; and
(c) comparing the determined expression to expression of the nucleic acid in a cell or tissue sample that is not contacted with the test compound,
wherein a difference in expression of the nucleic acid when the cell or tissue sample is contacted with the test compound indicates that the test compound can be used to treat schizophrenia.

4. A method of claim 3, wherein the levels of expression of a plurality of nucleic acids in the cell or tissue sample are determined, said plurality of nucleic acids are selected from the group consisting of SEQ ID NOS: 1-249.

5. A method of claim 4, wherein the levels of expression of at least fourteen nucleic acids in the cell or tissue sample are determined, said nucleic acids are selected from the group consisting of SEQ ID NOS: 1-249.

6. A method of claim 4, wherein the levels of expression of at least twenty-eight nucleic acids in the cell or tissue sample are determined, said nucleic acids are selected from the group consisting of SEQ ID NOS: 1-249.

7. A method of claim 4, wherein the levels of expression of at least forty-two nucleic acids in the cell or tissue sample are determined, said nucleic acids are selected from the group consisting of SEQ ID NOS: 1-249.

8. A kit for diagnosing schizophrenia in an individual, said kit comprising:
a plurality of nucleic acid probes, wherein each of said probes specifically hybridizes to a nucleic acid selected from the group consisting of SEQ ID NOS: 1-249.

9. A kit for diagnosing schizophrenia in an individual, said kit comprising:
a plurality of primer pairs, wherein each of said primer pair specifically amplifies a nucleic acid selected from the group consist of SEQ ID NOS: 1-249.

10. A method for monitoring a therapeutic response in an individual undergoing treatment for schizophrenia, said method comprising:
(a) determining expression, in the cell or tissue sample, from said individual of at least one nucleic acid selected from the nucleic acids of SEQ ID NOS: 1-249; and
(b) comparing the determined expression to the expression of the nucleic acid in a cell or tissue sample, from a individual who does not have schizophrenia
wherein a similar level of expression of the nucleic acid in the cell or tissue sample obtained from the individual undergoing treatment for schizophrenia relative to the level of expression of the nucleic acid in the cell or tissue sample obtained from the individual who does not have schizophrenia indicates a therapeutic response.
